(19) United States
(12) Patent Application Publication
(10) Pub. No.: US 2003/0059763 A1
(43) Pub. Date: Mar. 27, 2003

(54) Immunoglobulin class switch recombination

(76) Inventors: Andrew Saxon, Santa Monica, CA (US); Ke Zhang, Los Angeles, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/770,169

(22) Filed: Jan. 26, 2001

(51) Int. Cl. C12Q 1/00

(52) U.S. Cl. 435/4

(57) ABSTRACT

This invention concerns class switch recombination (CSR) substrates and assays that permit dissection of the mechanisms involved in CSR and are powerful tools in drug discovery.

This invention was made with Government support under Grant Nos. AI40551 and AI15251, awarded by the National Institutes of Health. The Government has certain rights in this invention.

### BACKGROUND OF THE INVENTION

1. Field of the Invention

The present invention concerns immunoglobulin (Ig) class switch recombination. More particularly, the invention concerns novel class switch recombination assays, and switch recombination systems, vectors and cell lines used in performing such assays.

2. Description of the Related Art

Immunoglobulin (Ig) class switching is a critical step in the generation of the diversified biological effector functions of the antibody response. Ig class switching allows expression of a variety of isotypes of Ig with different effector functions and maintains antigen specificity. Ig class switching involves non-homologous DNA recombination between two IgH switch (S) regions through a process known as class switch recombination (CSR) (Zhang et al., Regulation of class switch recombination of the immunoglobulin heavy chain genes. In: Immunoglobulin Genes, Second Edition, T. Honjo and F. W. Alt, eds. 1995, pp 235-265; Snapper et al., Immunity 6:217-223 (1997); Stavnezer et al., Current Topics in Microbiol. & Immunol. 245:127-168 (2000)). CSR involves a region of the upstream Ig locus rearranging with a downstream targeted S region. CSR results in a switch of expression from the upstream isotype to the downstream isotype. The intervening DNA is excised as circular DNA. Signals are important for achieving isotype switching. Cytokine-signaling is important in determining the specificity of Ig CSR. Cytokine-directed switching depends on the ability of cytokines to selectively induce and regulate IgH gernline transcription of the downstream gene. An “accessibility model” has been proposed for Ig CSR (Stavnezer et al., Proc. Natl. Acad. Sci. USA 85:7704-7708 (1988); Zhang et al., 1995, supra, and the references therein). This model proposes that a given cytokine induces transcriptional activity through a specific IgH locus to allow accessibility of the Ig switch machinery for CSR. In knock-out mice with deletion cytokine response elements for regulation of germline transcription, CSR to that specific locus is blocked. (Jung et al., Science 259:984-987 (1993); Zhang et al., J. Immunol. 152:3427-3435 (1994)). However, the cytokine responsive IgH locus control elements likely have functions other than driving transcription across the locus, e.g. LPS-inducible transcriptional activity through an Ig S region per se is not sufficient for efficient CSR (Xu et al., Proc. Natl. Acad. Sci. USA 90:3705-3709 (1993); Bottaro et al., EMBO J. 13:665-674 (1994)). While the inability to produce processed germline transcripts (or switch transcripts) in these mutant mice could account for a failure to undergo efficient CSR (Lorenz et al., Science 267:1825-1828 (1995)), this does not test the possibility that IgH locus control elements play a critical role in CSR by providing effects in addition to the transcriptional activity. The second signal that is important for CSR is through CD40 stimulation. CD40 stimulation is involved in activation of switch recombination.

To investigate the cellular processes involved with CSR, various plasmid- and retrovirus-based vectors with Ig switch DNA have been developed (Ott et al., EMBO J. 6:577-587 (1987); Leung and Maizels, Proc. Natl. Acad Sci. USA 89:4154-4158 (1992); Daniels and Lieber, Proc. Natl. Acad Sci. USA 92:5625-569 (1995); Ballantyne et al., Int. Immunol. 7:963-974 (1997); Borggrefe et al., J. Biol. Chem. 273:17025-17035 (1998); Kinoshita et al., Immunity 9:849-858 (1998); Stavnezer et al., J. Immunol. 163:2028-2040 (1999); Zhang and Cheah, Clin. Immunol. 94:140-151 (2000)). In particular, the role of DNA sequences, regulatory elements, and transacting factors in CSR have been investigated after transfection of the designed vectors into cells. Although important insights resulted through these approaches, dissection of CSR processes was limited.

Accordingly, there is a need for further CSR assays that would mimic one or more steps of the complex class switch recombinational processes, and would thus facilitate the dissection and understanding of CSR. Such systems would be powerful tools to identify components involved in Ig CSR, including the components of the putative switch recombinase complex and the participants in the signal transduction pathways leading to the activation of the switch recombinase, which are presently largely unknown.

### SUMMARY OF THE INVENTION

The present invention is based on the development of simple and efficient class switch recombination assays that permit dissection of the mechanisms involved in CSR and thereby provide insights that could not be gained through earlier approaches. The new assays find utility in the identification of participants and events involved in Ig CSR, including IgE CSR, and are, therefore, believed to be powerful tools in drug discovery, including the development of drugs for the treatment of allergic diseases associated with IgE production.

More specifically, the invention is based on the development of a new switch substrate which, upon recombination results in the expression of a reporter gene (GFP in the Examples) in living cells. This system shows high efficiency with up to or more than 50% of cells undergoing recombination.

Accordingly, in one aspect, the invention concerns an isolated nucleic acid molecule comprising

(a) a first class switch region (S₁) sequence of an upstream immunoglobulin locus under transcriptional control of a first promoter;

(b) a second class switch region (S₂) sequence of an immunoglobulin locus downstream of S₁ under transcriptional control of a second promoter, wherein said S₂ sequence serves as a region-specific substrate for class switch recombination (CSR);

(c) a nucleic acid encoding a reporter molecule interposed between the S₁ and S₂ sequences in reverse transcriptional orientation, and

(d) a promoter, downstream of the nucleic acid encoding said reporter molecule, allowing the expression of the reporter molecule only following switch recombination between the S₁ and S₂ sequences.

In another aspect, the invention concerns a switch vector comprising the foregoing nucleic acid molecule, and a recombinant host cell stably transfected with such vector.

In yet another aspect, the invention concerns a method of monitoring immunoglobulin (Ig) class switch recombination (CSR), comprising

(a) providing a switch vector comprising

(i) a first class switch region (S₁) sequence of an upstream immunoglobulin locus under transcriptional control of a first promoter;

(ii) a second class switch region (S₂) sequence of an immunoglobulin locus downstream of S₁ under transcriptional control of a second promoter, wherein the S₂ sequence serves as a region-specific substrate for CSR;

(iii) a nucleic acid encoding a reporter molecule interposed between the S₁ and S₂ sequences in reverse transcriptional orientation, and

(iv) a promoter, downstream of the nucleic acid encoding the reporter molecule, allowing the expression of the reporter molecule only following switch recombination between the S₁ and S₂ sequences;

(b) stably transfecting a mammalian cell with the switch vector; and

(c) monitoring the expression of the reporter molecule in the mammalian cell, wherein such expression indicates CSR.

In a further aspect, the invention provides an in vitro CSR assay comprising the steps of

(a) providing a switch vector comprising, under transcriptional control of a promoter and in natural transcriptional orientation,

(i) a first class switch region (S₁) nucleotide sequence of an upstream Ig locus;

(ii) a second class switch region (S₂) nucleotide sequence of an Ig locus downstream of said upstream Ig locus; and

(iii) a reporter gene nucleotide sequence encoding a reporter molecule, interposed between the S₁ and S₂ sequences;

(b) incubating the switch vector with a cell-free nuclear extract from Ig-producing cells; and

(c) detecting deletion of the reporter gene.

In a particular embodiment, deletion of the reporter gene is detected following transformation of the switch vector into recombinant host cell, e.g. E. coli cells. The reporter gene may, for example be a lacZ gene, deletion of which can be detected by counting the white colonies obtained after transformation, in the presence of isopropyl-β-D-thiogalactoside (IPTG) and 5-bromo-4-chloro-3-indolyl-β-D-galactoside (X-gal).

The immunoglobulin-producing cells may, for example, be B lymphocytes, and the S₁ and S₂ sequences preferably contain G-rich, tandemly repetitive sequences. In a particularly preferred embodiment, the B cells are activated, for example by CD40.

### BRIEF DESCRIPTION OF THE DRAWINGS

FIG. 1A is a schematic diagram of substrate switch recombination (SSR) events in the prototype of the switch construct, XF-1. Once the SSR takes place, the Sμ joins to the Sγ2 with the intervening DNA between the Sμ and Sγ2 being looped-out and excised. The excised DNA ends are joined to form the circular DNA, in which the IRES-GFP expression unit is under the control of the RSV-LTR. Splicing donor and acceptor sites have been inserted as shown and provide for the formation of the uniform IRES-GFP transcripts. In the case of an inversion between Sμ and Sγ2, the IRES-GFP expression unit would then be under the transcriptional control of the pCMV and GFP would also be expressed. The long straight arrows represent transcriptional orientation, whereas the short arrows indicate the promoter transcriptional initiation sites. In the foregoing description, IRES: Internal Ribosomal Entry Site; pCMV: CMV promoter; pRc: pRc/RSV LTR enhancer/promoter; pSV; SV40 promoter; Sd: splicing donor site; Sa: splicing acceptor site; PA: polyA site.

FIG. 1B shows the frequency of SSR in various switch constructs induced by IL-4, CD40 mAb or IL-4 plus CD40 mAb. On the left, a schematic diagram of the various constructs is given, while on the right, the percent of switched cells (GFP positive) is shown. The numbers represent the mean values±standard deviation (SD) from three independent experiments with the clones indicated in the parentheses. The cells were cultures for 6 days with medium, IL4 (1 ng/ml), CD40 mAb (1 μg/ml) or both. Th results given in the FIG. 1B are described in details in the Examples.

FIG. 2 shows flow cytometric analysis for GFP positive cells in stably transfected Ramos 2G6 cells. Ramos 2G6 cells (1×10⁵ cells/ml) transfected with the XF-1 and XF-5a constructs were cultured for 6 days in the presence of medium, IL-4 (3 ng/ml), CD40 mAb (3 μg/ml) or IL-4 plus CD40 mAb. The upper left panel shows the gating to exclude dead cells that accumulated in stimulated cultures. The upper rights panel shows the negative control for GFP expression from XA-1 construct. The data represent one of the four similar experiments.

FIG. 3 shows Southern blot analysis of PCR amplified switch fragments from SSR in XF-5a.1 cells.

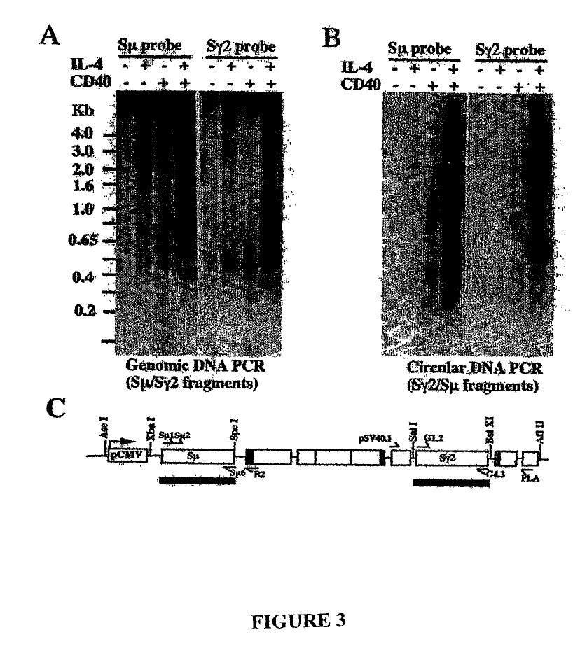

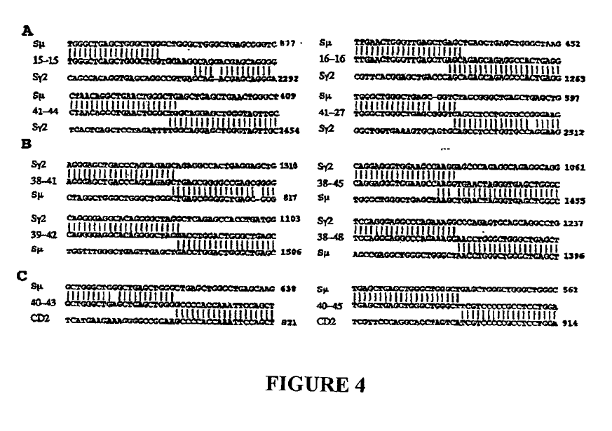

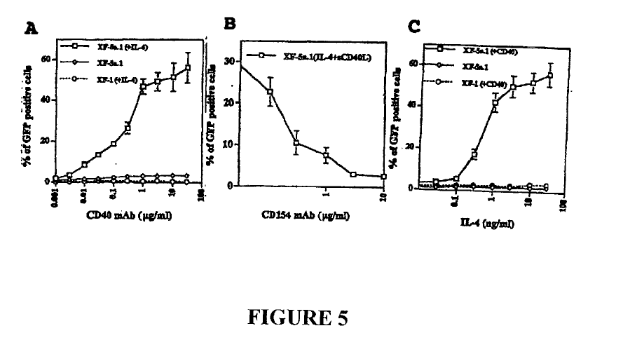

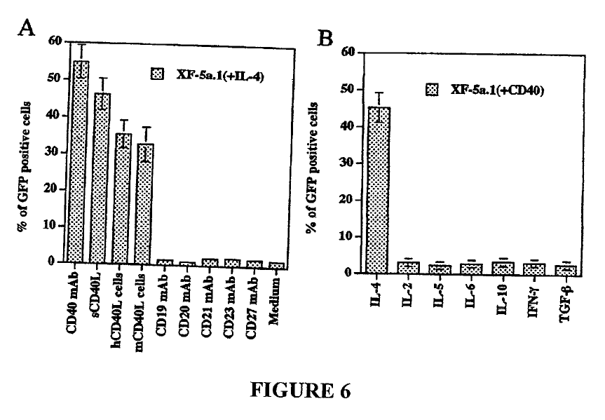

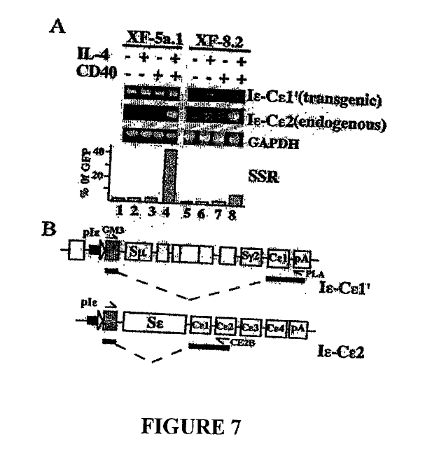

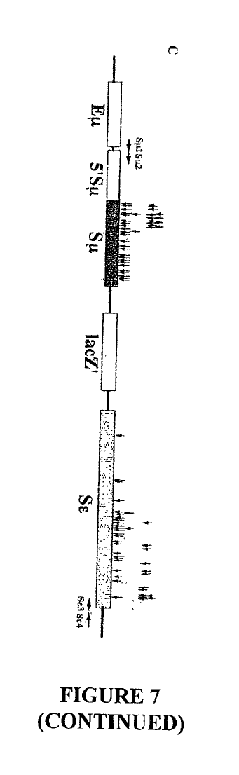

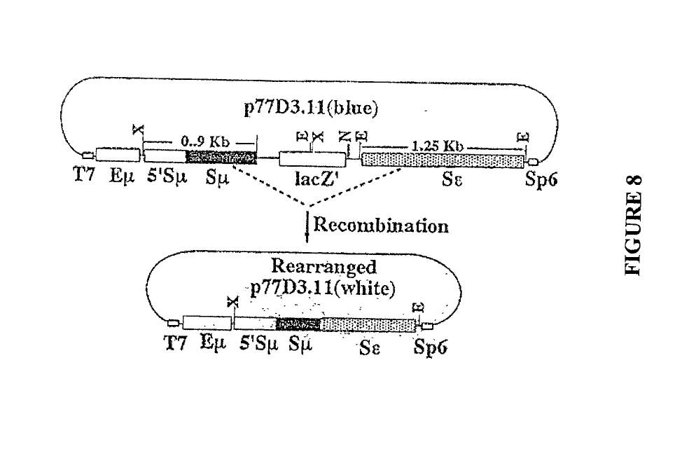

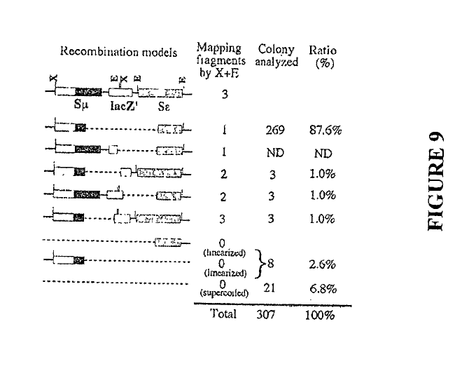

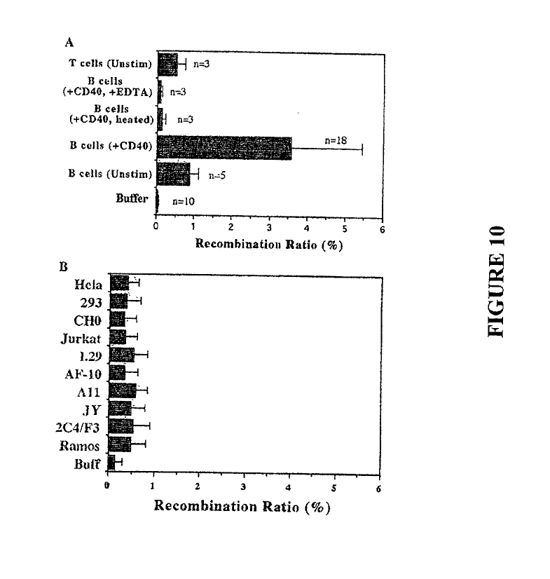

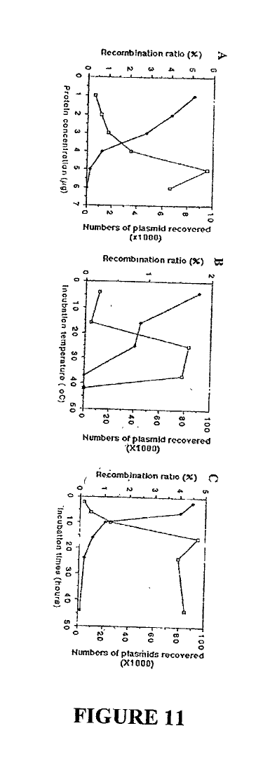

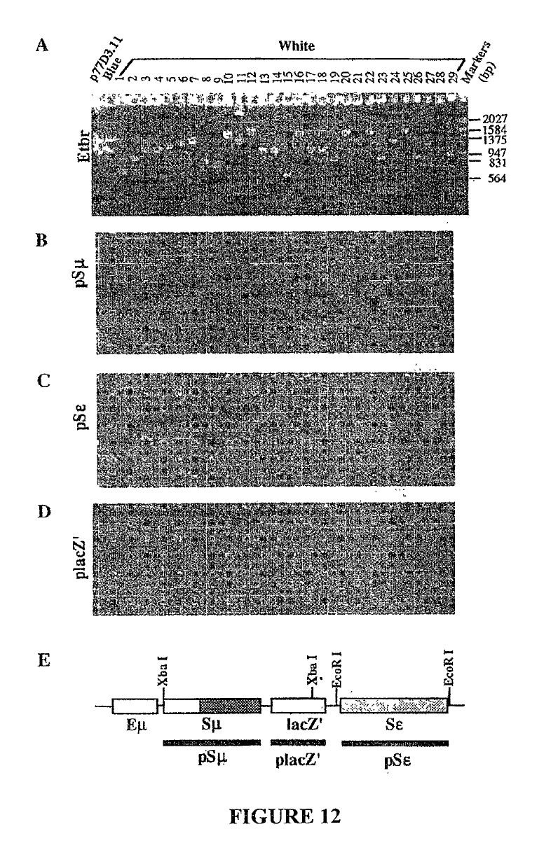

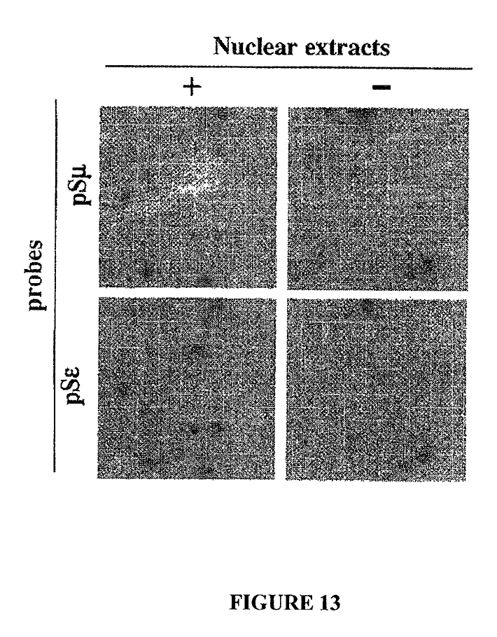

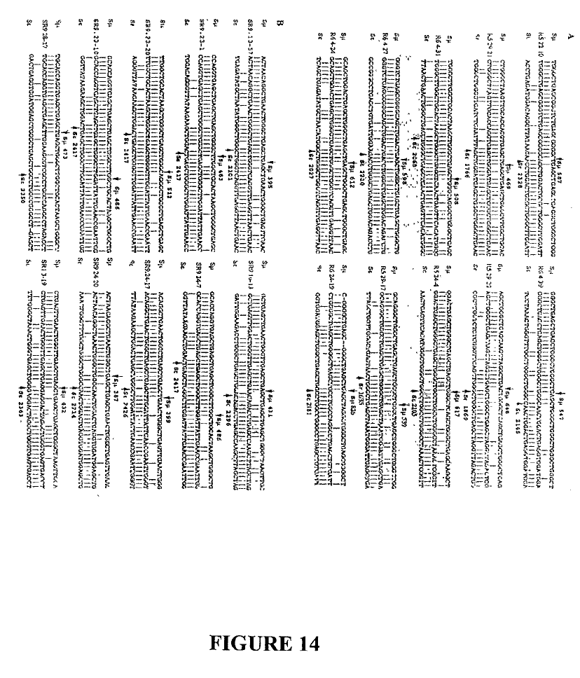

A. Southern blot analysis for the switch fragments amplified from genomic DNA. Genomic DNA (0.1 μg) from unstimulated and stimulated XF-5a.1 cells was subjected to PCR to detect switch fragments using the primers diagrammed in FIG. 3C. PCR products were electrophoresed and transferred for hybridization to the Sμ and Sγ2 probes as shown. The results represent one of the three similar experiments performed.

B. Southern blot analysis for the switch fragments amplified from deleted circular DNA. DNA represented the circular DNA fraction (0.1 μg) from unstimulated and stimulated XF-5a.1 cells was subject to PCR for switch fragments. PCR products were hybridized with Sμ and Sγ2 probes as shown. The results represent one of the two similar experiments performed.

C. Diagram of the PCR strategy and probes used for Southern blot analysis. The primers Sμ1-PLA and Sμ2-G4-3 were used to amplify the Sμ/Sγ2 fragments for first round and second round PCR respectively. The primers B2-pSV40.1 was used to amplify Sγ2/Sμ fragments for first round while the primers Sμ6-G1.2 were used for second round PCR. The Xba I—Spe fragment from XF-5a was used as Sμ probe while Sal I—Bst XI fragment formed the Sγ probe.

FIG. 4 shows nucleotide sequences surrounding the recombination breakpoints from the PCR clones hybridizing to either the Sμ or Sγ2 probes. The homologous sequences in the breakpoints are in bold. The numbers in the end of each sequence represent the position of last nucleotide that serves as the reference for the position of the recombination breakpoints.

A. The sequences surrounding the breakpoints in clones representing Sμ-Sγ2 recombination sites in genomic DNA derived from the switch construct. A total of 13 clones were sequenced and four of them are shown.

B. The sequence surrounding the breakpoints in clones representing the excised circular DNA resulting from Sγ2-Sμ recombination. A total of 6 clones were sequenced and four of them are shown.

C. The sequence surrounding the breakpoints from the clones representing the Sμ-CD2 recombination in genomic DNA. A total of 3 clones were sequenced and two of them are shown.

FIG. 5 shows CD40- and IL-4-dependent SSR in Ramos 2G6/XF5a.1.

A. Dose-response of SSR to CD40 mAb stimulation in Ramnos 2G6/XF-5a.1 cells. The frequency of SSR with the various indicated amount of CD40 mAb in the presence or absence of IL-4 (1 ng/ml) from four experiments.

B. Inhibition of SSR by anti-CD40L mAb (CD154). The frequency of SSR with different concentrations of CD154 mAb (CD40L) in the presence of IL-4 (1 ng/ml) plus sCD40L (0.5 μg/ml) from two experiments.

C. Dose-response of SSR to IL-4 stimulation. The frequency of SSR with various IL-4 doses as indicated in the presence or absence of CD40 mAb (1 μg/ml) from three experiments are presented.

FIG. 6 shows the specificity of SSR in the XF-5a.1 cells. The cells were cultured for 6 days with IL-4 (1 ng/ml) or CD40 mAb (1 μg/ml) plus the reagents indicated and analyzed for GFP positive cells by FACS.

A. SSR in Ramos 2G6/XF-5a.1 cells is CD40-specific. CD40 mAb, sCD8-CD40L and all antibodies were added at 1 μg/ml. Cells expressing human CD40L or murine CD40L were irradiated with 8000 Rads before addition to the cultures at a 1:1 ratio with Ramos 2G6/XF-5a.1 cells. The data shows the mean values from triplicate cultures from one of the three similar experiments.

B. SSR in Ramos 2G6/XF-5a.1 cells is IL-4-specific The data represent the mean values from triplicate cultures from one of the three similar experiments in which L-4 and other cytokines (5 ng/ml) was added.

FIG. 7 shows the transcriptional activity vs. SSR in Ramos 2G6/XF-5a.1 versus Ramos 2G6/XF-8.2 under the various conditions. The cells were cultured in the presence of IL-4 (1 ng/ml) and CD40 mAb (1 μg/ml) for 48 hours following by RNA preparation and RT-PCR. Total RNA (2 μg) was used for reverse-transcription and cDNA derived from 0.2 μg of RNA was subject to PCR amplification using the primers diagrammed in the FIG. 7B. The ratio of SSR from the cultures (assayed at day 5) is shown in the bottom. These results represent one of the four similar experiments performed. Diagrams of the formation of the “germ line” transcripts Iε-Cε1′ from the transgenes and the Iε-Cε2 from the endogenous IgH ε locus are shown in the FIG. 7B. The arrows represent the positions of the primers used for PCR.

FIG. 8 is a diagram of plasmid p77D3.11 and in vitro switch recombination in this switch substrate. The Eμ, Sμ, lacZ′ gene, and Sε fragments are constructed in the natural transcriptional orientation. The lacZ′ gene is flanked by the 5′ Sμ and 3′ Sε fragments, as shown. The tandemly repeated sequence regions in Sμ and Sε and indicated by heavy shading and light shading, respectively. Restriction endonucease sites that are utilized in the manipulation and analysis of this plasmid are shown. Recombination between Sμ and Sε deleted the lacz′ gene, resulting, after transformation, in white colonies in the presence of IPTG and X-gal. X, XbaI; E, EcoRI; N, NotI.

FIG. 9 shows the patterns of the recombination in p77D3.11. The possible recombination patterns are diagrammed on the left, whereas the recombination frequencies of each pattern detected are listed on the right. Among all the clones analyzed, 97.6% of the recombination events occurred between the two S DNA. The recombination frequency that occurred between two S regions was significantly higher than other types of recombination (P<0.0001). ND=not determined.

FIG. 10 shows the frequency of the in vitro switch recombination with nuclear extracts. The recombination ratio was calculated by scoring the recombined colonies (white) vs. the non-recombined colonies (blue) from the same transformation dish. The bars represent the average value from the number of experiments (n), as indicated above the error bars. The error bars represent one standard deviation. (A) Recombination frequencies from primary B and T cells with 5-μg nuclear extracts incubated for 16 hours under the conditions indicated. (B) Recombination frequencies from the cell lines with 1-μg nuclear extracts incubated for 16 hours.

FIG. 11 shows the results of the optimization of the switch recombination assay in vitro. The open squares represent the recombination ratio, whereas the filled squares represent the plasmid recovery rates. The nuclear extracts for the experiments were from CD49 mAb-stimulated tonsillar B cells. The plasmid recovery rates were calculated by scoring the transformed plasmids from a given dish. The final numbers were multiplied by the dilution factors for each reaction. The data represent the average value from three experiments, (A) the protein concentration (from 1 to 5 μg) vs. recombination ratio and rates or plasmid recovery, (B) the incubation temperature vs. recombination ratio and rate of plasmid recovery and (C) the incubation time vs. recombination ratio and rate of plasmid recovery.

FIG. 12 shows the results of the analysis of recombinational clones by restriction mapping and Southern blot hybridization. (A) Restriction mapping of clones by XbaI+EcoRI. Twenty-nine randomly picked recombined clones (white colonies), as well as one non rearranged clone (blue colony), were digested with XbaI+EcoRI. The digested plasmids and products were resolved in 1% agarose gel and stained by EtBr. (B) The restriction-mapped products shown in A were hybridized with probe pSμ. (C) The restriction-mapped products shown in A were hybridized with probe pSε. (D) The restriction-mapped products shown in A were hybridized with probe placZ′. The weak signals on the blot were due to the incomplete strip of the previous hybridization to pSμ and pSε as the sequence analysis conferred that there were no lacZ′ gene sequences in those weak positive clones. (E) Diagram of thc probes used in the hybridization shown in B-D.

FIG. 13 shows in situ hybridization for detection of the positive colonies for Sμ and Sε products derived from PCR amplification. The blots were individually hybridized to pSμ and pSε, as diagrammed in the figure legend for FIG. 11. The three strong positive dots in the periphery of each blot are hybridization-positive controls and serve for blot orientation. They contain the non rearranged plasmid p77D3.11. The blots derived from the extracts (+) can be seen to have at least five positive colonies to both pSμ and pSε, while only the hybridization controls are seen in the absence of the extracts.

FIG. 14 shows the nucleotide sequences surrounding the retained recombination breakpoints. The recombinational breakpoints are indicated by arrows with the referenced nucleotide position according to the published sequences (Lyon and Aguilera, Mol. Immunol. 34:209-219 (1997)). The sequences homologous between Sμ and Sε are bold. (A) Nucleotide sequences surrounding the recombination breakpoints derived from recombination assay-derived clones. (B) Nucleotide sequences surrounding the recombination breakpoints derived from direct PCR-generated clones without bacterial transformation. (C) Summary of location of al the recombination breakpoints defined from recombination assay and PCR amplification assigned to Sμ and Sε regions is p77D3.11. The arrows in the top row represent the recombination breakpoints defined from PCR amplification, whereas those in the lower row represent the recombination breakpoints defined from recombination assay. The primer positions for amplification of the Sμ-Sε recombinational products are also indicated.

### DETAILED DESCRIPTION OF THE PREFERRED EMBODIMENT

I. Definitions

Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “immunoglobulin” (Ig) is used to refer to the immunity-conferring portion of the globulin proteins of serum, and to other glycoproteins, which may not occur in nature but have the same functional characteristics. The term “immunoglobulin” or “Ig” specifically includes “antibodies” (Abs). While antibodies exhibit binding specificity to a specific antigen, immunoglobulins include both antibodies and other antibody-like molecules that lack antigen specificity. Native immunoglobulins are secreted by differentiated B cells termed plasma cells, and immunoglobulins without any antigen specificity are produced at low levels by the lymph system and at increased levels by myelomas. As used herein, the terms “immunoglobulin,” “Ig,” and grammatical variants thereof are used to include antibodies, and Ig molecules without antigen specificity.

Native immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The main Ig isotypes (classes) found in serum, and the corresponding Ig heavy chains, shown in parentheses, are listed below:

IgG (γ chain): the principal Ig in serum, the main antibody raised in response to an antigen, this antibody crosses the placenta;

IgE (ε chain): this Ig binds tightly to mast cells and basophils, and when additionally bound to antigen, causes release of histamine and other mediators of immediate hypersensitivity; plays a primary role in allergic reactions, including hay fever, asthma and anaphylaxis; and may serve a protective role against parasites;

IgA (α chain): this Ig is present in external secretions, such as saliva, tears, mucous, and colostrum;

IgM (μ chain): the Ig first induced in response to an antigen; it has lower affinity than antibodies produced later and is pentameric; and

IgD (δ chain): this Ig is found in relatively high concentrations in umbilical cord blood, may be an early cell receptor for antigen, and is the main lymphocyte cell surface molecule.

Antibodies of the IgG, IgE, IgA, IgM, and IgD isotypes may have the same variable regions, i.e. the same antigen binding cavities, even though they differ in the constant region of their heavy chains. The constant regions of an immunoglobulin, e.g. antibody are not involved directly in binding the antibody to an antigen, but exhibit various effector functions, such as participation of the antibody in antibody-dependent cellular toxicity (ADCC).

Some of the main antibody isotypes (classes) are divided into further sub-classes. IgG has four known subclasses: IgG1 (γ1), IgG2 (γ2), IgG3 (γ3), and IgG4 (γ4), while IgA has two known sub-classes: IgA1 (α1) and IgA2 (α2).

A light chain of an Ig molecule is either a κ or a λ chain.

During development, stem cells formed in a yolk sac, liver, or bone marrow migrate to lymph nodes and the spleen, where individual cell lines undergo clonal development independent of antigen stimulation. Most cells initially produce IgM, and later switch to the production of IgG, IgE, or IgA isotypes. Once B cells are released into the circulation and reach peripheral lymphoid tissues, they are capable, if stimulated by antigen, of differentiating into plasma cells that produce antibody specific for the antigen encountered.

As used herein, “class switching” or “isotype switching” means a change in the phenotype of an Ig-producing cell. Ig class switching is a critical step in the generation of the diversified biological effector functions of the antibody response. For example, as mentioned above, B cells initially produce primarily IgM, a phenotype change into the production of IgG, IgE or IgA is an “isotype switch” or “class switch.” Class switching, as used herein, includes two steps: the first step is the provision of trans-spliced transcripts to act as bridging templates for conforming genomic immunoglobulin DNA, and the second step is switch recombination that results in the production of switch circles and rearrangement of genomic Ig DNA to allow production of a different Ig (antibody). In particular, Ig class switching involves DNA recombination between two IgH switch (S) regions through a non-homologous recombination, a process known as class switch recombination (CSR). This process leads to the rearrangement of the S region of the upstream Ig locus to a downstream targeted S region and results in the expression of the downstream isotype. The intervening DNA is looped-out, and excised as the switch circular DNA.

The term “switch region” or “S” region is used to refer to a nucleotide sequence composed of tandem repeat sequences that occur in nature 5′ to the Ig heavy chain constant region and function in intrachromosomal class switching, i.e., recombination of DNA sequences encoding specific portions of Ig heavy chain constant regions, and variants of such sequences retaining the class switching function of the native sequences. Examples of specific switch regions are disclosed, for example in Mills et al., J. Immunol. 155:3021-3036 (1995). “Switch region” or “S” region includes both full-length switch sequences of native immunoglobulins, and recombinant and synthetic sequences that contain substitutions, insertions, deletions and/or other modifications relative to a native Ig S region, provided that they retain the function of providing a substrate for CSR.

The term “nucleic acid” refers to polynucleotides such as deoxyribonucleic acid (DNA), and, where appropriate, ribonucleic acid (RNA). The term also includes, as equivalents, analogs of either DNA or RNA made from nucleotide analogs, and as applicable, single (sense or antisense) and double-stranded polynucleotides. An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structure such as polyamides.

A “reporter gene” is a gene whose expression results in a detectable signal. The term “detectable signal” is used in the broadest sense and includes any change between the expressed and non-expressed state of the gene, such as color change, or a detectable label, e.g. a fluorescent, radioactive, enzymatic (such as, urease, alkaline phosphatase, or peroxidase), or other, e.g. avidin/biotin label. In the case of enzyme tags, colorimetric indicator substrates are known which can be employed to provide a means visible to the human eye or spectrophotometrically. Accordingly, practically any detectably labeled gene can serve as a reporter gene. A typical example of reporter genes is Green Fluorescent Protein (GFP) gene, the expression of which can be monitored in stably transfected living cells by single- or dual-color flow cytometry (FACS). Another suitable reporter gene is the lacZ gene, the expression of which results in blue colonies in transformed host cells, in the presence of isopropyl-β-D-thiogalactoside (IPTG) and 5-bromo4-chloro-3-indolyl-β-D-galactoside (X-gal). Thus, the extent of expression of the lacZ gene can be monitored by counting the white (non-expressed) vs. blue (expressed) colonies of transformed host cells. Other suitable reporter genes include, for example, β-galactosidase, luciferase, secreted alkaline phosphatase (SEAP), just to mention a few.

A “host cell” includes an individual cell or cell culture which can be or has been a recipient of any vector of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo with a vector comprising a nucleic acid of the present invention.

The term “promoter” means a nucleotide sequence that, when operably linked to a DNA sequence of interest, promotes transcription of that DNA sequence.

II. Modes of Carrying Out the Invention

A. Preferred Embodiments of Cell-based Class Switch Recombination Assay

In one aspect, the invention concerns a novel, real-time substrate switch recombination system (SSR), which allows the direct and quantitative detection of individual events of switch recombination in living cells. The key component of this system is a switch construct that comprises upstream and downstream class switch regions, flanking a reporter gene inserted in reverse transcriptional orientation, which is not expressed in germline configuration of the construct. This model mimics Ig class switch recombination (CSR), being CD40 and IL-4 dependent, preferring GC-rich regions, and characterized by non-homologous recombination.

Several class switch regions (S regions) have been characterized, including the murine Sμ, Sε, Sα, Sγ₃, Sγ₁, Sγ₂ₐ and Sγ_{2b} switch regions and human Sμ and Sγ switch regions (Mills et al., (1995), supra.) For example, the murine Sμ region is about 3 kb and can be divided into a 3′ region with sequences [(GAGCT)ₙGGGGT]ₘ, wherein n=1-7 and m=150 (Nikaido et al., Nature 292:845-848 (1981)), and a 5′ region in which these two pentamers are interspersed with the following heptamer sequence: (C/T)AGGTTG (Marcu et al, Nature 298:87-89 (1982)). The human Sμ sequence is different in that the heptamer sequence is distributed throughout the region (Takahashi et al., Cell 29:671-679 (1982); Mills et al. (1995) supra). All S sequences contain multiple copies of the pentarneric sequences GAGCT and GGGGT, and the pentamers ACCAG, GCAGC, and TGAGC are also commonly found in S regions (Gritzinacher, Crit. Rev. Immunol. 9:173-299 (1989)). In addition, thc foregoing heptameric repeat is also commonly found in native S regions. All these regions/repeats, and similar regions from other murine or human S regions will be referred to as “GC rich” regions or repeats.

As noted before, S regions used in the switch constructs of the present invention can be naturally occurring sequences, which may be cloned directly from an Ig locus, e.g. a human or murine Ig locus, or may produced by recombinant and/or synthetic means. The S regions may also differ from the native S sequences by nucleotide alterations, e.g. deletions, substitutions, insertions, and/or other modifications, relative to a native S region, provided that the altered S region retains is functionality, i.e. ability to facilitate recombination. If desired, modified S regions can be designed to have an improved (enhanced) ability to facilitate recombination, compared to a native sequence

Each class switch region is under control of a promoter, which may be a non-inducible/constitutive promoter, such as pCMV or pSV40, a strong transcriptional promoter, such as pIμ/Eμ, tobacco mosaic virus promoter (pTMV), promoters from cauliflower mosaic virus (35S/pCaMV), promoter for Elongation Factor 1alpha (pEF-1α), Epstein-Barr Virus promoter (BC-R2), and promoter for Human T cell Leukemia Virus (HTLV). Similarly, any strong transcriptional promoter, including those listed above, may be inserted in the switch construct downstream of the reporter gene to control its expression upon class switch recombination.

In a preferred embodiment, the switch constructs of the present invention the upstream switch (S) region is under control of a cytokine-inducible promoter, which selectively determines the accessibility of the S region DNA to switch recombinase and is, thus, required for efficient class switch recombination. An example of such cytokine-inducible promoters is the IL-4 inducible Iε promoter, the use of which is illustrated in the Examples. The Iε promoter sequence preferably also includes the Evolutionarily Conserved Sequences (ECS), Iε exon sequences and the Iε exon splicing donor site. These sequences provide for the ability to undergo high level inducible CSR following stimulation by IL-4, and optionally other factors involved in CSR. While the cytokine-inducible promoter, e.g. Iε promoter, renders switch DNA recombinationally accessible for CSR (modeled by CSR in the experimental system of the present invention), optimal CSR (SSR) requires both cytokine-inducible promoter activity and strong transcriptional activity.

In another preferred embodiment, the switch constructs of the present invention contain a GC-rich downstream switch region (S₂), serving as a region-specific substrate for class switch recombination (CSR). In the present cell-based assay, the switch recombinase machinery preferentially targets GC-rich substrate S DNA, closely resembling the situation at the intrinsic IgH locus.

The upstream and downstream Ig S regions (S₁ and S₂) may be native, naturally occurring sequences, which may be isolated from their native source, or produced by recombinant and/or synthetic means. Alternatively, the S regions may be variants of the native sequences, provided that they retain the ability to participate in class switch recombination. In a preferred embodiment, S₁ and S₂ are native sequences from Ig heavy chain genes.

The switch constructs of the invention are incorporated into expression vectors, containing and capable of expressing such constructs in appropriate recombinant host cells. By way of example, without limitation, such vectors may be pCMV, adenoviral, adeno-associated viral, retroviral vectors, pUC and derivatives thereof, M13 and derivatives thereof, SV40, and the like.

According to the cell-based assay of the invention, DNA of the switch constructs is stably transfected into recombinant host cells, preferably immunoglobulin-producing lymphoid cells, e.g. B cells, or cells with antibody-producing potential (e.g. stem cells). In some embodiments, the B cell is a primary B cell, such as a human primary B cell, or a B cell line, such as, for example, Ramos, BL-2, JY, CL-01 or 2C4/F3. Numerous methods of stable transfection are known to the skilled worker in the field, including transfection by the calcium phosphate coprecipitation technique; electroporation; electropermeabilization; liposome-mediated transfection; ballistic transfection; biolistic processes including microparticle bombardment, jet injection, and needle and syringe injection; or microinjection.

The detection of class switch recombination depends on the nature of the reporter gene used. For example, the expression of GFP in cell lines stably transfected with the switch vectors herein can be measured by either single- or dual-color flow cytometry (FACS Core laboratory, UCLA), and the data analyzed with FCS express software (De Novo Software Inc., Thornhill, Ontario, Canada), as illustrated in the Examples. Methods for detecting the expression of other reporter genes are also well known in the art.

B. Preferred Embodiments of Cell-Free Assay

The in vitro recombination system disclosed herein employs cell-free nuclear extracts from Ig-producing cells or cells with antibody-producing potential, e.g. stem cells to detect CSR between switch (S) regions, preferably human S regions in a cell-free system. Antibody-producing cells and cell lines, and cells with antibody-producing potential are well known in the art and include, for example, primary B cells, hybridoma cell lines expressing antibodies, embryonic stem cells (e.g. a murine embryonic stem cell), and the like.

The availability of the S region sequences has been discussed above. As noted before, S regions can be naturally occurring sequences, which may be cloned directly from an Ig locus, e.g. a human or murine Ig locus, or may produced by recombinant and/or synthetic means. The S regions may also differ from the native S sequences by nucleotide alterations, e.g. deletions, substitutions, insertions, and/or other modifications, relative to a native S region, provided that the altered S region retains is functionality, i.e. ability to facilitate recombination. If desired, modified S regions can be designed to have an improved (enhanced) ability to facilitate recombination, compared to a native sequence. The S region sequences preferably retain the tandemly repetitive G-rich sequences found in native mammalian, e.g. human S regions. The preferential targeting of G-rich, tandemly repetitive S region sequences in this assay system by the nuclear extract recombination activity resembles primary switch recombination events, where the switch circular DNA preferentially targets such sequences.

The reporter gene can be any gene the expression of which provides a detectable (and preferably quantifiable) signal. A particularly suitable reporter gene is a lacZ gene, which makes it possible to monitor DNA recombination between two S regions by blue-white selection. CSR deletes the lacZ gene, resulting, after transformation, in the formation of white colonies in the presence of IPTG (isopropyl-β-D-thiogalastoside) and X-gal (5-brorno-4-chloro-3-indolyl-β-D-galactoside), whereas the non-recombined plasmid gives blue colonies since the interposed lacZ gene remains intact. The recombination activity in this assay can be enhanced by CD40 stimulation, as illustrated in the examples.

C. Uses of the Invention

The technology disclosed herein represents of platform from which one can dissect the molecular events involved in human immunoglobulin isotype switching. The vectors developed can be used under cell-free conditions, or in permanently transfected cell lines, such as Ramos cell lines, to undergo events equivalent to immunoglobulin class recombination. The vectors are such that they can be modified to represent immunoglobulin class switching to the various human heavy chain loci (IgG1-4, IgA1-2, and IgE). Thus, by modifying the switch vectors to represent different isotypes, one can then dissect the general features of isotype switching that are common between the isotypes and more critically, unique features that will show differential ability to control the production of different human immunoglobulins.

The vectors provide for a read-out which allow one to either block, add or stimulate specific molecular product-gene products and examine their effect on switching to a specific immunoglobulin heavy chain type. The molecules so identified are targets for altering human immunoglobulin isotype switching. Once a specific molecule involved in isotype switching has been identified, using the cell-based assay, one can screen the transfected cells with compounds using large throughput systems to look for small molecule/drug candidates that will affect the target molecules. Furthermore, any compound shown to bind these target molecules can be tested to see if they will in fact block switching, using the constructs of the present invention.

In addition to testing possible gene or gene product candidates as potential drug targets, the assays of the present invention provide a much broader technology to screen libraries to look for candidate genes involved in isotype switching. Once identified, such genes (and their products) become the targets for drug discovery. To accomplish this, DNA libraries can be constructed from cell driven switch to different immunoglobulin isotypes. These libraries can then be transfected into the cell line, or used in the cell frec system to see if they contain genes whose products will drive or block isotype switching. The switching read-out system will be monitored by an appropriate means of detection, such as by flow cytometry for switching, by detecting the expression of the reporter gene, e.g. GFP. Cells that undergo switching once following transfection will be carrying a gene or genes of interest. They can be expanded and then the genes identified. This allows for the identification of the whole repertoire of molecules that are directly involved in general isotype switching, as well as those molecules that are involved in switching to a specific isotype or show relative specificity for one or more isotypes. Such molecules then become drugs themselves, or become the targets for drug development.

In a particular aspect, the present invention enables the identification of molecules capable of channeling isotype switching away from an undesired isotype towards a more benign isotype.

For example, IgE-mediated allergy reactions result from the binding of an allergen (such as found in pollen, dander or dust) to IgE that is bound to the surface of basophils and mast cells. Such binding causes cross-linking of the underlying receptors, and the subsequent release of pharmacological mediators, such as histamine, causing common symptoms of allergy. At present, common allergic diseases (allergic rhinitis, allergic asthma, atopic dermatitis, stinging insect allergic reactions) are estimated to affect about 20-30% of the population.

Treatment of allergic disease is complex and variable, but can be divided into three major approaches. Environmental controls are designed to eliminate or at least minimize exposure to the allergen. Symptomatic drug therapy is required in the control of most common allergies. The drugs used for this purpose include anti-histamines and systemic or topical corticosteroids and sympathomimetics. Immunotherapy of allergy is accomplished by administration of gradually increasing doses of allergen over a period of years with the hope that the patient will develop increasing tolerance to the allergen. The precise mechanism of immunotherapy is still unknown, however, clinical improvement in some patients correlates well with the level of IgG-blocking antibodies, which presumably act by binding the allergen and preventing its interaction with mast cell-bound IgE. However, immunotherapy is not consistently effective for all sufferers of allergic symptoms. Further, the immunotherapy regimen can be costly, requires significant discipline on the part of the patient for success, and has attendant risk of local and systemic reactions. Alternative strategies include steroid injections that generally suppress the whole immune system and have a host of other undesirable side effects and hence, by definition, put the patient at risk.

The present invention provides assays suitable for identifying molecules that can inhibit or block the production of an undesired Ig isotype, e.g. IgE, and can thus be used in the prevention and/or treatment of allergic diseases.

Alternatively present invention provides assays suitable for identifying molecules that can enhance the production of an desired Ig isotype, e.g. IgA, and can thus be used in the prevention and/or treatment of infectious diseases. For example, approximately 1:2000 persons is unable to produce IgA antibodies, antibodies that play a critical role in the defense of mucosal surfaces such as the sinuses, large airways, genital tract and gastrointestinal tract. Failure to produce IgA may thus lead to recurrent and chronic sinusitis, bronchitis and gastrointestinal problems. Currently there is no way to replace passively replace IgA so that treatment for symptomatic IgA subjects relies upon good hygiene, vaccination against infections where possible (e.g. influenza, streptococcal pneumoniae) and frequent antibiotics. Molecules that will drive IgA production may provide treatment for IgA deficient subjects. Additionally, they will be useful with vaccine administration as a way of driving a more IgA (mucosal oriented) response which will be advantageous against infectious organisms that primary gain entry at mucosal surfaces (e.g. HIV or influenza).

In the screening assays of the invention, the library screened can, for example, be a chemical library, a combinatorial chemistry library, a combinatorial biologically-encoded library (e.g., a SELEX library or a phage display library), or a collection of protein variants. The compounds screened specifically include small organic molecules, which typically are less than about 2000 Da in size, more commonly less than about 1000 Da in size, preferably less than about 500 Da in size, more preferably less than about 250 Da in size, most preferably less than about 200 Da in size. Other compounds that can be screened in accordance with the present invention are peptides, or polynucleotides, including RNA and DNA molecules, antisense nucleic acid, etc.

Further details of the invention are illustrated by the following non-limiting Examples. The Examples are provided so as to provide those of ordinary skill in the art with a complete disclosure and description of how to make and use the products and methods of the invention, and are not intended to limit the scope of what the inventors regard as their invention. Efforts have been made to insure accuracy with respect to numbers used (e.g. amounts, temperatures, etc.) but some experimental errors and deviation should be accounted for. Unless indicated otherwise, parts are parts by weight, temperature is in degrees C, and pressure is at or near atmospheric. The disclosures of all citations throughout this specification are hereby expressly incorporated by reference.

III. Examples

### EXAMPLE 1

A Real-time Switch Recombination System

a. Construction of the Switch Vectors

The switch vectors were constructed as follows:

pXF: A 1.8 Kb human Sγ2 fragment was amplified by PCR with the 5′ primer (TTGTCCAGGCCGGCAGCATCACCGGAG) (SEQ ID NO: 1) and the 3′ primer (ACTCCTCAGTGGGATGGCC TCTACACTCCCT) (SEQ ID NO: 2) (Mills et al, J. Immunol. 155:3021-3036 (1995)) and cloned into a switch vector p77D3.11 (Zhang and Cheah, 2000, supra) to replace the Sε fragment. This generated a shuttle vector p77D4. A Xba I+Spe I fragment that contained the Sμ-pUC 18 linker-Sγ2 was inserted into the Nhe I site in pEGFP-N2 vector (Clonetech, Palo Alto, Calif.) to create pN2-17. The Bgl II+Not I fragment containing EGFP was deleted from the vector to create pN2-17 XA-1. A 2 Kb Sal I+Xho I fragment containing IVS-IRES-EGFP-BGHpA from pIRES-EGFP (Clonetech, Palo Alto, Calif.) was cloned into the polylinker sites of pN2-17 XA-1 between Sμ and Sγ2 in an opposite transcriptional orientation to create pN2-17 XA-2. A 5′-splicing donor site from the first intron of the human beta-globin gene (including the branch point) was amplified from pCI-neo vector (Promega Inc, Madison, Wis.) with primers B (CTAGAAGCTTTATTGCGGTAGT) (SEQ ID NO: 3) and C (CGACAAGCTTAGTTTCTATTGGTC) (SEQ ID NO: 4) and cloned into the Hind III site of pRc/RSV vector (Invitrogen, San Diego, Calif.) to create pRc/RSV-Sd. The Afl II site in pRc/RSV-Sd was removed by Afl II digestion, blunting and self-ligation. A Sal I+Xho I fragment containing RSV LTR and a splicing donor site from pRc/RSV-Sd was inserted into the Xho I site in pN2-17 XA-2 in the reverse transcriptional orientation to create pXF.

pXF-1: The Sal I−Bgl II fragment from IVS in pXF was replaced with PCR amplified 325 bp SV40 promoter sequence. A PCR amplified 425 bp fragment containing the Cε1 exon and its 5′fianking sequences, including slicing acceptor site, was inserted in the position 3′ to Sγ2.

pXF-5a: A PCR amplified 356 bp fragment containing Iε exon and its 5′ promoter region (pIε) was inserted into Xho I and Xba I sites in pXF-1.

PXF-8: The CMV promoter in pXF-5a was deleted by removal of the Ase I and Xho I fragment.

pXF-2a: A PCR amplified 449 bp fragment containing the human Iμ promoter/Eμ enhancer was cloned into the Xho I and Xba I site in pXF-1.

pXF-2b: The CMV promoter in pXF-2a was deleted by removal of the Ase I and Xho I fragment.

pXF-6a: The 1.8 Kb Sγ2 fragment in pXF-5a was replaced with a 1.25 Kb Sε fragment from p77D3.11 (Zhang and Cheah, 2000, supra).

pXF-6b: The 1.8 Kb Sγ2 fragment in pXF-5a was replaced with a 1.1 Kb human CD2 cDNA (Sewell et al, Proc. Natl. Acad. Sci. USA 83:8718-8722 (1986)) amplified from PCR.

b. General Description

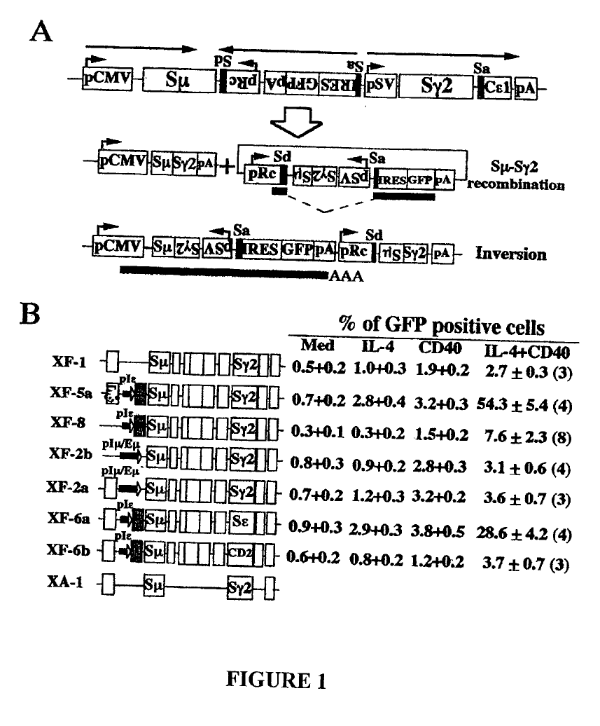

The backbone of our switch substrate contained 1.6 Kb of the human Sμ (Zhang and Cheah, 2000, supra) and 1.8 Kb of the human Sγ2 regions (Mills et al, 1995, supra) under the transcriptional control of the CMV (pCMV) and the SV40 promoters (pSV40) respectively. A RSV LTR enhancer/promoter (pRC/RSV-LTR) and a GFP gene under the control of an internal ribosome entry site (IRES) were interposed between Sμ and Sγ2 in the reverse transcriptional orientation (FIG. 1A). Thus, the GFP gene will not be expressed within the gennline configuration of the construct. Detection of switch recombination between the Sμ and Sγ2 in the construct is based on the mechanism of the deletional recombination that excises and deletes the intervening DNA sequences between the Sμ and Sγ2. This excised sequence including the IRES-GFP expression unit forms an extrachromosomal circular DNA that is now under the transcriptional control of the pRSV-LTR (FIG. 1A). The RNA splice donor and acceptor sites permit uniform expression of the GFP from the excised circular DNA (FIG. 1A). Since the GFP gene is expressed only after the switch recombination (recombination or inversion, FIG. 1A) between the Sμ and Sγ2, switch recombination can be monitored in the cultured living cells in “real-time” by fluorescence microscopy. Furthermore, the frequency of switch recombination can be quantitatively measured by flow cytometry.

To test the potential roles of DNA sequences that may control or regulate the processes involved in switch recombination, the DNA fragments of interest were engineered into the basic switch construct (XF-1) to generate a series of modified switch constructs as diagrammed in the FIG. 1B. All the modified constructs were transfected into Ramos 2G6 cells as stable constructs to establish the switch cell lines. The integrity and copy number of the constructs were determined by Southern Blot analysis and PCR. Only subclones that contain a single copy of the construct were used. To distinguish the substrate switch recombination described here from switch recombination occurring at the intrinsic IgH loci (e.g., CSR), the former is abbreviated as SSR.

### EXAMPLE 2

Study of Immunoglobulin Class Switch Recombination (Ig CSR)

a. Materials and Methods

1. Cell Lines, Culture, and Transfection

The human B lymphoma cell line Ramos 2G6 (ATCC, Rockville pike, Md.) and its subclones, L cells stably transfected with human CD40L (CD154) provided by D. Rawlings, UCLA), and CHO cells stably transfected with murinc CD40L (Immunex Inc, Seattle, Wash.) were maintained and cultured in complete PRMI 1640 (Zhang et al, 1994, supra). One million cells in 0.2 ml were transfected with 10 μg plasmid DNA by electroporation (200V, 0.975 μF). Genicitin (G418) was added to cultures two days later for clonal selection and the levels increased over four week to concentrations that were previously determined by dosage titration for the different cell lines.

2. Cytokines and anti-CD4 mAb

Anti-CD40 mAb G28.5 was produced from a hybridoma cell line obtained from ATCC. IL-2, IL-4, IL-5, IL-6, IL-10, TGF-β, IFN-γ were purchased from R & D system. The soluble CD154-CD8 fusion protein (sCD40L) was kindly provided by Dr. G-H. Cheng (UCLA). The anti-CD154 mAb was from Phaffingen (San Diego, Calif.).

3. Polymerase Chain Reaction (PCR)

PCR and real-time PCR (RT-PCR) were performed as described previously (Zhang et al, 1994, supra; Zhang and Cheah, 2000, supra). For amplification of the Sμ-Sγ2 products, genomic DNA from the stably transfected Ramos 2G6 cells was amplified with PCR Kit for GC-rich DNA (Clonetech, Palo Alto, Calif.). PCR was carried with the primer pair Sμ1 (ACTCAGATGGCTAAACTGAGCCTAAGCT) (SEQ ID NO: 5) and PLA (ATGTTTCAGGTTC AGGGGGAGGTGTG) (SEQ ID NO: 6) for the first round of amplification, and Sμ2 (GAGCCTAGACTAAC AGGCTGAACT) (SEQ ID NO: 7) and G4.3 (ACTCCTCAGTGGGATGGACTCACACTCCC T) (SEQ ID NO: 8) for the second round amplification. For amplification of the circular DNA, the primer B2 (AAGCTTTATTGCGGTAGTTT ATCACAGT) (SEQ ID NO: 9) and pSV40-1 (CCAAGATCTCCAGGCAGGCAGAAGTAT) (SEQ ID NO:10) were used for first round and primer Sμ6 (CCCAACTAGTCTTAGCCTGATACAACCTG) (SEQ. ID NO: 11) and G1.2 (TTGTCCAGGCCATC AGCATCACTGGAG) (SEQ ID NO: 12) were used for second round PCR. For RT-PCR, the upstream primer GM3 (AGCTGTCCAGGAACCCGACAGGGAG) (SEQ ID NO: 13) and downstream primer PLA were used to amplify Iε-CH1′ transcripts. GM3 and primer Cε2B (GTTGATAGTCCCTGGGGTGTA) (SEQ ID NO: 14) were used to amplify the Iε-CH2 from endogenous Iε germline transcripts.

4. Fluorescence-Activated Cell Sorting (FACS) Analysis

The expression of GFP in cell lines stably transfected with switch vectors following various stimulated culture conditions was measured by either single- or dual-color flow cytometry (FACS Core laboratory, UCLA). The data were analyzed with FCS express software (De Novo software Inc, Thornhill, Ontario, Canada).

b. Results

1. High Level SSR is Inducible in the Construct Carrying the IL-4-Inducible Iε Promoter Sequence

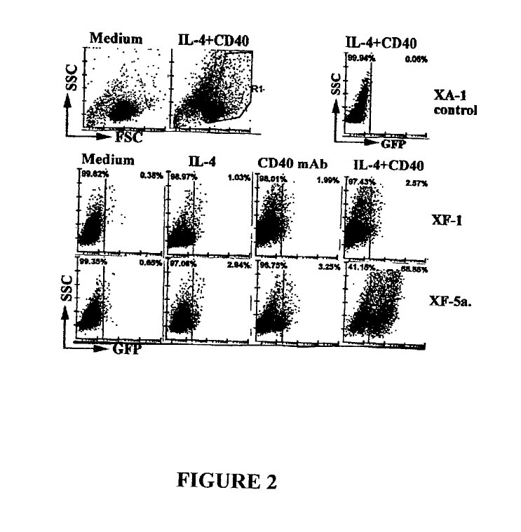

To test the efficiency of the SSR in the constructs, we cultured the stable transfected Ramos 2G6 cells with IL-4, CD40 mAb or a combination of both. The SSR rates for construct XF-1 in which the transcriptional activity is controlled by pCMV increased from 0.5% in medium (spontaneous SSR) to 1.9% upon CD40 mAb stimulation, a significant but rather limited increase (p<0.05) (FIG. 1B and FIG. 2). IL-4 had marginal effects on increasing the SSR frequency by itself and did not significantly enhance CD40 mAb driven SSR in XF-1 (FIG. 1B and FIG. 2). Thus CD40 stimulation was able to minimally enhance SSR in XF-1 while IL-4 did not. In contrast, IL-4 plus CD40 mAb stimulation markedly enhanced SSR in XF-5a; a construct containing defined Iε sequences. Up to 54.3% of the cells from four independent Ramos 2G6/XF-5a clones expressed GFP following optimal IL-4 plus CD40 mAb simulation (FIG. 1B, FIG. 2). IL-4 or CD40 mAb alone had limited effects on the SSR in these XF-5a cell lines (FIG. 1B and FIG. 2). The strong induction of the SSR by IL-4 plus CD40 stimulation in XF-5a was not due to the proliferation effects of this combination as synergistic effects on proliferation were not observed. In fact, IL-4 plus CD40 induce higher rates of cell death compared with IL-4 or CD40 mAb stimulation alone in this cell line (FIG. 2 and data not shown). Thus IL-4 markedly potentiated CD40 mAb induced SSR in XF-5a as opposed to XF-1. The structural differences between XF-1 and XF-5a are that XF-5a contains a 356 bp DNA fragment of the IL-4-inducible Iε promoter starting from 161 nucleotides upstream of the most common initiation site of Iε exon (Gauchat et al, J. Exp. Med. 172:463-473 (1990)). This includes the Evolutionarily Conserved Sequences (ECS), Iε exon sequences and the Iε exon splicing donor site. These sequences provide for the ability to undergo high level inducible SSR following stimulation by IL-4 and CD40 mAb in XF-5a.

2. SSR is Reflected by GFP Expression and Represents Non-homologous DNA Recombination

To confirm that the GFP expression was derived from the SSR excised circular DNA in the transfected constructs, genomic and circular DNA were subjected to PCR amplification to detect Sμ/Sγ2 and Sγ2/Sμ switch fragments. Genomic DNA from IL-4 plus CD40 mAb stimulated XF-5a showed far more PCR amplified DNA bands that hybridized to either Sμ or Sγ2 probes than from medium controls or cells stimulated with IL-4 or CD40 mAb alone (FIG. 3A). Correspondingly, PCR products representing deleted circular DNA were abundant in IL-4 plus CD40 mAb stimulated cells, rare in IL-4 or CD40 mAb stimulated cells and not detectable from the unstimulated cells (FIG. 3B). These results directly reflected the frequency of GFP positive cells induced by IL-4 and/or CD40 mAb stimulation. Cloning and sequence analysis of the PCR products revealed that they represented non-homologous recombination between the two S DNA regions, a characteristic of Ig CSR (FIG. 4A, 4B). Taken together, these results demonstrate that the GFP expression correlates with the events of switch recombination derived from the constructs.

3. SSR is CD40 Dependent

To determine the nature of the SSR dependence on CD40 stimulation, Ramos 2G6/XF-5a.1 cells were cultured separately with CD40 mAb, soluble CD40 ligand (sCD40L), human CD40L-expressing L cells, murine CD40L-expressing CHO cells and various cytokines. In the absence of IL-4 stimulation, SSR in XF-5a.1 was induced by CD40 mAb in a dose-dependent fashion, although to a relatively low frequency, going from 0.7% to 3.2% (FIG. 1B and FIG. 5A). Administration of higher concentrations of CD40 mAb did not further increase the SSR frequency (FIG. 5A). SSR was also induced by various CD40L reagents including human sCD40L, human CD40L, and murine CD40L (FIG. 5B, FIG. 6A and data not shown). SSR was not induced by anti-CD19, anti-CD20, anti-CD21, anti-CD23, or anti-CD27 (FIG. 6A). These results indicate that CD40 stimulation is sufficient to enhance SSR and the effect is CD40 specific.

SSR dose-dependency on CD40 stimulation, in the presence of IL-4, was clearly demonstrated in FIG. 5A and 5B. The GFP expression level increased from about 1% to greater that 50% with increasing concentrations of CD40 mAb. The isotype matched control mAb had no effect (data not shown). The role of CD40 in the SSR was further confirmed by addition of anti-human CD40L mAb to the cultures, which blocked sCD40L-driven GFP expression in a dose-dependent fashion (FIG. 5B).

4. SSR in the Constructs Carrying the Iε Promoter is IL-4-Dependent and Specific

The data in FIG. 5C show that in addition to being CD40 dependent, IL-4 effects on switch recombination in XF-5a were dose-dependent in the presence of CD40 mAb with the GFP expression going from 3.3% without IL-4 to a plateau around 50% at 3 ng/ml. Other cytokines tested, including IL-2, IL-5, IL-6, IL-10, TGF-β, and IFN-γ, either alone or in combination with CD40 mAb, did not increase the SSR in XF-5a.1 or XF-1 transfected cells (FIG. 6B and data not shown). Thus the cytokine-dependent SSR in XF-5a.1 is IL-4 specific and dependent FIG. 6B). The ability of IL-4 to promote the SSR in XF-5a but not in XF-1 is presumably exerted through IL-4-inducible Iε promoter activity.

5. The IL-4-Inducible Iε Promoter, the CMV Promoter and the Iμ Promoter, but Not the pCMV or the Iμ/Eμ Promoter/Enhancer, Controls the Efficiency of SSR

Next we examined the effects of the Iε promoter, the CMV promoter and the Iμ promoter/Eμ enhancer on SSR. As previously shown, under the sole transcriptional control of pCMV (XF-1), only low level SSR (2.7%) could be induced by IL-4 plus CD40 mAb whereas the same stimuli drive highly efficient SSR in XF-5a.1 that contains the IL-4 inducible Iε promoter (FIG. 1B). In XF-5a.1, in the absence of IL-4, the frequency of SSR following CD40 stimulation alone was again low (3.2%), even though the S DNA was under the control of the pCMV (FIG. 1B and FIG. 2). Similarly, when the DNA was under the sole control of the Iμ promoter/Eμ enhancer (construct XF-2b), efficient SSR did not result from CD40 mAb and/or IL-4 stimulation (FIG. 1B). Furthermore, replacement of the IL-4 inducible Iε promoter in XF-5a with the Iμ promoter/Eμ enhancer (XF-2a) still did not result in highly efficient SSR in response to CD40 mAb plus IL-4 (FIG. 1B). The inability to induce efficient SSR in XF-2a and XF-2b is not due to our cloning of a non-functional Iμ promoter/Eμ enhancer as in a transient transfection assay, the cloned Iμ promoter/Eμ enhancer strongly drove the GFP expression from a promotcrless GFP vector (data not shown). These results demonstrate that while the pCMV- and the Iμ promoter/Eμ enhancer drive transcription through the S DNA, they only provide for a basal level of SSR.

6. Efficient SSR is Selectively Induced by IL-4 but Not pCMV-driven Transcriptional Activity: Transcriptional Activity Itself Does Not Correlate with SSR

The role of transcriptional activity vs. SSR was further investigated using Ramos 2G6/XF-5a.1. As the XF-5a construct contains the Iε exon and a Cε1 exon (referred to as Cε1′) that is able to form processed transcripts through RNA splicing, transcriptional activity of the construct can be semi-quantitatively determined by measuring the Iε-Cε1′ transcripts by RT-PCR (FIG. 7). Thus the proportional transcriptional activity contributed by the pCMV and/or the IL-4-inducible Iε promoter in the construct can be accessed. There was a high level of constitutive transcriptional activity through the S DNA in the XF-5a.1 cells under pCMV and pSV40 control with high levels of the Iε-Cε1′ transcripts produced in the unstimulated culture (FIG. 7A, lane 1). However this pCMV transcriptional activity fail to lead to a high frequency of SSR (<0.5%) in the presence of the culture medium, IL-4 or CD40 stimulation alone (FIG. 1B, FIG. 2 and FIG. 7). Stimulation with IL-4 plus CD40 mAb gave >50 fold higher SSR but enhanced the level of processed Iε-Cε1′ transcripts slightly (less than one fold) over that driven by the pCMV (FIG. 7A, Lane 4). Likewise, CD40 mAb or IL-4 alone minimally increased the pCMV-driven transcriptional activity (FIG. 7A). A similar outcome was also observed in other XF-5a subclones (data not shown). These data clearly show that the rate of transcription driven by the pCMV does not correlate with the SSR in XF-5a. In contract, the IL-4 inducible Iε promoter which plays a critical role in rendering the S DNA recombinationally accessible for highly efficient SSR does so without significantly enhancing the pCMV-driven transcriptional activity across the S DNA.

7. IL-4-Inducible Iε Promoter Activity Alone is Not Able to Induce Optimal SSR

The high level of SSR in XF-5a.1 driven by IL-4 plus CD40 is achieved under the control of both the pCMV and the Is promoter. To test whether the IL-4-inducible Iε promoter itself in the presence of IL-4 and CD40 is sufficient to drive efficient SSR, we deleted the pCMV from XF-5a to create XF-8 (FIG. 1B). In the absence of the pCMV in XF-8, the transcriptional activity through the upstream Sμ was absent as indicated by the lack of the Iε-Cε1′ transcripts (FIG. 7A, Lane 5). IL-4 alone, which had a weak effect on inducing the 1s gernline transcripts from the endogenous IgH ε gene, also induced low level expression of the Iε-Cε1′ transcripts from the XF-8 construct in Ramos 2G6 cells (FIG. 7A, lane 6). The level of IL-4-induced Iε-Cε1′ transcripts was not increased further by CD40 mAb in XF-8 and was much lower than that driven by the pCMV in XF-5a (FIG. 7A, lane 4 and 8). Interestingly, while stimulation with IL-4 plus CD40 was able to induce XF-8 to switch reasonably well (7.6%±2.3%, N=8), the levels were still clearly lower than in XF-5a under the same conditions (FIG. 1B and FIG. 7). Transcription driven by the pCMV did not correlate with SSR as IL-4 plus CD40 driven SSR rates in XF-8 was more than a fold higher than that in CD40 stimulated XF-5a in spite of transcription in XF-8 being far less than that in pCMV driven XF-5a (FIG. 7). These results provide two key insights; the Iε promoter itself is not sufficient to induce optimal SSR, (e.g. to a level compatible to that in XF-5a), and strong transcriptional activity is necessary, although not sufficient, for the optimal SSR directed by the Iε promoter.

The level of Iε-Cε1′ transcripts induced by IL-4 was not significantly enhanced by CD40 mAb in XF-5a and M-8 (FIG. 7A, lands 4 and 8). In contrast, the level of IL-4-induced endogenous Iε germline transcripts (indicated as Iε-Cε2) was markedly increased by CD40 stimulation in the same Ratnos 2G6 cells under the same conditions (FIG. 7A). This indicates that in terms of germlinc transcripts, IL-4 induced Iε promoter activity in the transgenes (XF-5a and XF-8) and the endogenous ε locus respond to CD40 stimulation differently.

8. Substrate Switch Recombination Machinery Preferentially Targets GC Rich S DNA

To test whether typical GC rich S DNA is required for the efficient SSR, we modified the XF-5a construct by replacing the Sγ2 with 1.2 Kb of Sε, a GC rich sequence, or with 1.1 Kb of CD2 cDNA, non-GC rich DNA, thereby creating MF-6a (Sε) and XF-6b (CD2) respectively (FIG. 1B). As with XF-5a, the Sε based XF-6a was able to switch efficiently following IL-4 plus CD40 stimulation (FIG. 1B). In contrast, the SSR frequency in XF-6b with CD2 as the target “switch region” was much lower (3.7%±0.7%). On the other hand, the SSR in XF-6b was clearly induced by IL-4 plus CD40 stimulation as compared to XF-6b alone or stimulated with IL-4 or CD40 (FIG. 1B). Cloning and sequencing analysis of the PCR amplified switch fragments revealed that they were non-homologous recombination (FIG. 4C and data not shown). Thus in our switch system, as with the intrinsic IgH loci, the switch recombinase machinery preferentially targets GC rich S DNA, but such DNA is not absolutely required for SSR, and non-GC rich DNA sequences can be used as the recombination substrate, albeit poorly.

c. Discussion

We have developed a powerful experimental approach for investigating Ig CSR employing a novel switch substrate which, upon recombination, results in GFP expression in living cells. This system shows high efficiency with up to or >50% of cells undergoing recombination. Furthermore, this system is sensitive, convenient and performed in “real-time” manner related to the events of switch recombination. Using flow cytometric measurement of GEP expression, this novel model is capable of quantitatively measuring SSR.

A major obstacle in the field of Ig CSR is that the assay systems previously reported were not robust enough to allow the individual events of switch recombination to be directly and/or quantifiably detected in living cells. The experimental system reported herein overcomes most of those problems. Our SSR model is not only efficiently inducible, but also a genome-integrated system using stable transfection with the switch constructs. In contrast to transient transfection assays which maintain the switch constructs in an extrachromosomal states outside the chromatin structure (Leung and Maizels 1992, supra; Daniels and Lieber, 1995, supra; Cherry and Baltimore, Proc. Natl. Acad. Sci. USA 96:10788-10793 (1999)), our integrated switch system will have the features of chromosomal structure resembling the native structural environment for switch recombination. Thus our approach is particularly useful in defining 1) the nature of the changes induced by cytokine-inducible promoters that render S DNA recombinationally accessible for efficient switch recombination, 2) the DNA conformation required for efficient switch recombination, 3) the signal transduction pathways leading to the activation of Ig class switch recombinase, and 4) the components that directly or indirectly involved in the activation the putative switch recombinase.

The data presented in this Example provide some interesting insights that could not be revealed by previously described systems. Most importantly, these data demonstrate that the cytokine-inducible promoters, exemplified the IL-4-inducible Iε promoter, play an unique role in efficient SSR, a role that can not be replaced by strong transcriptional promoters such as CMV promoter and/or the Iμ promoter/Eμ enhancer for efficient SSR (see below).

1. Accessibility of S Region DNA to Ig Class Switch Recombinase Requires more than Transcription: The Specific Role for IL-4-Inducible Iε Promoter in SSR

The “accessibility model” of Ig CSR proposes that I promoter(s) induced transcription through S DNA renders the S DNA recombinationally accessible to the putative switch recombinase for CSR. While this general concept has strong support from experiments in vivo, in vitro and with knock-out mice (Jung, et al, 1993, supra; Zhang et al, EMBO J. 12:3529-3537 (1993); Zhang et al, 1995, supra; Stavnezer, 2000, supra), the nature and role of the I promoter (s) driven transcriptional activity in CSR have not been clearly defined. That I promoter(s) may provide functions for CSR beyond the transcriptional activity is suggested by evidence that 1) efficient CSR does not result from the replacement of native cytokine-inducible promoters with other promoters that actively transcribe S DNA (Xu et al, 1993, supra, Bottaro et al 1994, supra) and 2) some viral or other constitutively-activated/inducible promoter-controlled switch constructs show low rates of SSR (Ballantyne et al, 1997, supra; Kinoshita et al, 1998, supra; Stavnezer et al, 1999, supra). By comparing the rote of the IL-4-inducible Iε promoter with the pCMV and Iμ promoter/Eμ enhancer, we now show that IL-4-inducible promoter efficiently facilitates SSR mediated by CD40 activated SRA whereas strong promoter activities from the pCMV and Iμ promoter/Eμ enhancer do not. These results define that simply having strong transcriptional activity is insufficient to render S DNA optimally accessible for switch recombinase. In contrast, our results show that transcriptional activity driven by the native cytokine-inducible promoter for Ig germline transcription plays a critical role in providing accessibility of S DNA for efficient SSR. Thus the accessibility of S DNA to the putative switch recombinase is selectively determined by the specific transcriptional activity driven by specific I promoters, such as IL-4-inducible Iε promoter, rather than by general transcriptional activity. These results explain why in mutant mice and switch constructs (Xu et al, 1993, supra, Bottaro et at 1994, supra; Ballantyne et al, 1997, supra; Kinoshita et al, 1998, supra; Stavnezer et at, 1999, supra) the highly active transcription through S DNA driven by non-cytokine inducible I promoters are not sufficient to promote efficient CSR. Our results can also explain why la exon targeted mutant mice in which the Iα exon was replaced with HPRT minigene but which maintained the endogenous TGF-β-inducible promoter, switched to the a locus as efficiently as wild-type mice. (Harriman et at, J. Clin. Invest. 97:477-487 (1996), Qiu et at, Int. Immunol. 11:37-45 (1999)).

By what possible mechanisms does an IL-4-inducible Iε promoter render S DNA appropriately accessible for efficient SSR? It is likely that sequence-specific transcriptional factor(s) and/or co-activator(s) that are driven by a given cytokine are essential for rendering S DNA appropriately accessible to the switch recombinase. Such cytokine (s) activated transcriptional factor (s), (e.g., IL-4 activated Stat6), may help assemble the SRA or participate in altering the chromatin structure in preparation for SRA. They would not be present in the transcriptional complex assembled by general promoters such as viral promoters, explaining why viral or certain constitutively activated promoters do not drive efficient CSR while maintaining strong transcriptional activity.

2. The role of the Strong Transcriptional Activity for Optimizing Efficient SSR Mediated by the Iε Promoter.

The finding that the Iε promoter itself confers the ability to induce Iε-Cε1′ transcripts but, in the absence of an additional transcriptional activity, is not sufficient to induce highly efficient SSR is striking but not surprising in light of the nature of in vivo CSR. It has been demonstrated that in addition to the cytokine-inducible I promoters, efficient in vivo CSR requires the activity of the 3′ α enhancer (Cogne et al, Cell 77;737-747 (1994)). Indeed that efficient SSR driven by the IL-4-inducible Iε promoter requires an additional transcriptional activity as observed in our switch constructs may well mimic the physiological conditions for CSR that requires 5′ and/or 3′ enhancers for efficient function. Thus sub-optimal SSR induced by the IL-4-inducible Iε promoter in XF-8 is likely due to the weak transcriptional activity driven by Iε promoter alone. The pCMV, which by itself is not sufficient to render S DNA accessible for efficient SSR, provides the Iε promoter with high level transcription with the resulting efficient SSR. This conclusion is supported by the recent findings that the 3′ α enhancer greatly enhances transcriptional activity driven by human γ3 and α promoters through enhancer-promoter interaction (Pan et al, Eur. J. Immunol. 30:1019-1029 (2000); Hu et al, J. Immunol. 164:6380-6386 (2000)). Enhanced transcriptional activities provided by the 3′ α enhancer may well correlate with their requirements for efficient CSR in vivo and is currently under the investigation in our laboratory since our experimental system provides a model to directly test the potential roles of the 3′ α enhancer in Ig CSR.

3. SRA is CD40 Dependent but Cytokine Independent

Signaling through CD40 is a critical step for CSR. CSR is abolished in both CD40- and CD40L-deficient mice and humans (Xu et al, Immunity 1:423-431 (1994); Aruffo et al, Cell 72:291-300 (1993); Kawabe et al, Immunity 1:167-178 (1994)). The requirement for CD40 signaling in CSR is believed to be due to its ability to induce and/or activate the putative switch recombinase as well as to synergize the cytokine-induced germline transcription. However, as successful CSR requires both cytokine and CD40 stimulation, whether the activation of the putative switch recombinase also requires cytokine stimulation in addition to CD40 had been unproven. Kinoshita et al. suggested that the activity of the putative switch recombinase required cytokines induction in addition to CD40 stimulation (Kinoshita et al, 1998). To directly test the requirements for activation of the SRA, we tested the effects of CD40 stimulation and cytokines on SSR because the proportional contributions of cytokines and CD40 effects on SSR are distinguishable from each other in our system.

Our results support the idea that CD40 stimulation itself is sufficient to induce or/and activate SRA. Thus in constructs lacking the Iε promoter, CD40 stimulation alone induces SSR with equal frequency to that induced by CD-40 plus IL-4 or a host of other cytokines. On the other hand, IL-4 drastically increases SSR in the constructs containing the IL-4-inducible Iε promoter in a cytokine specific and the Iε promoter-dependent fashions. These results indicate that IL-4 or other cytokines are not required for CD40 to activate SRA, at least in Ramos 2G6 cells. Therefore while CD40 can synergize with cytokine(s) in inducing germline transcription of the targeted locus for efficient CSR, the cytokine (s) (at least IL-4) does not appear to be required to activate SRA in the presence of CD40 stimulation. The low frequency of SSR in XF-5a induced by IL-4 alone (FIG. 1B and FIG. 2) can be attributed to the low level spontaneous SRA in this cell line, because IL-4 only potentiates the SSR in XF-5a but not in XF-1.

### EXAMPLE 3

Cell-free Recombination of Immunoglobulin Switch-Region DNA with Nuclear Extracts

a. Materials and Methods

1. Construction of the Model Plasmid

The model plasmid for in vitro recombination was constructed on the background of the pCRII vector (Invitrogen Corp., San Diego, Calif.). A 1.25-kb Sε fragment was generated by polymerase chain reaction (PCR) with a 5′ -primer (TGTCCCTTAGAGGACAGGTGGCCAA) (SEQ ID NO: 15) that corresponded to 2402-2378 of Sε (Mills et al., Nucleic acids res. 18:7305-7316 (1990)). The amplified fragment was cloned into the multiple clone sited of the lacZ gene in PCRII vector by TA cloning method as described (Zhang et al., J. Immunol. 152:3427-3435 (1994)). The insertion of the 1.25 kb Sε fragment disrupted the internal lacZ expression. A 0.91-kb fragment containing Sμ and its 5′ flank sequences was amplified with a 5′ primer (TCTAGACAAGGGGACCTGCTCATT) (SEQ ID NO: 16) that corresponded to 914-885 885 of Sμ (Mills et al., 1990, supra) and cloned into the EcoRV sited of the pBluescripts by TA cloning (Zhang et al., 1994, supra). A XBaI-XhoI fragment containing Sμ and its 5′ flank sequences excised from pBluescripts was inserted into the pCR II vector containing a 1.25-kb Sε fragment. An intact lazz gene (referred to as lacZ′ to distinguish the internal disrupted lacZ) amplified from pU18 vector was cloned into the ClaI-XhoI sited of the pCR II vector between 0.91-kb Sμ and 1.25-kb Sε fragments. A PCR-amplified 0.46-kb fragment containing human μ intronic enhancer sequences (Eμ) was inserted into the ApaI sites (Rabbitts et al., Nature 306:806-809 (1983)) (FIG. 8). All the fragments in the construct were determined to be in the normal transcriptional orientation by restriction endonuclease mapping and partial DNA sequence analysis. The resulting plasmid, designated as p77D3.11, was used as a model for in vitro S-S recombination.

2. Cell and Cell Lines

Human primary B and T lymphocytes were prepared and purified from human tonsils as described (Zhang et al., 1994, supra). The B cells were harvested for nuclear extract preparation following culture for 3 days with or without 0.1 μg/ml of CD40 monoclonal antibody G28.5 (Zhang et al., 1994, supra). Cell lines 2C4/F3 (provided by Dr. F. Finkelman), JY, A11 (provided by Drs. H. Yssel and J. deVries), I.29 (provided by Dr. J. Stavnzer), Ramos, AF-10, Jurkat, CHO, and Hela were maintained in complete RPMI 1640 (Zhang et al., 1994, supra). The 293 cell lines (from ATCC) were maintained in complete DMEM medium.

3. Preparation of Nuclear Extracts

Nuclear cell extracts from primary human B cells and cell lines were prepared by the Nonidet P-40 (NP-40) lysis method (Dyer and Herzog, BioTechniques 19:192-195 (1995)) with important modifications. Briefly, 10⁸ (10⁷ for cell lines) purified fresh human tonsillar B lymphocytes (Zhang et al., 1994, supra) were harvested following culture for 3 days with or without 0.1 μg/ml of anti-CD40 monoclonal antibody G28.5 (Zhang et al., 1994, supra) and were lysed with 1 ml of sucrose buffer (0.32 M sucrose, 3 mM CaCl₂, 2 mM magnesium acetate, 0.1 mM EDTA, 10 mM Tris-HCl, pH 8.0, 1 mM DTT, 0.5 mM PMSF) containing 0.5% (vol/vol) NL-40 (BRL, Gaithersburg, Mich.). The cells were lysed immediately and the intact nuclei were pelleted by centrifugation at 2400 rpm/5 min at 4° C. The nuclei were washed twice (10⁸ B cells/ml) with ice-cold sucrose buffer lacking NP-40. The pelleted nuclei were suspended in 50 μl/10⁸ B cells with low-salt buffer (20 mM Hepes, pH 7,6, 25% glycerol, 1.5 mM MgCl₂, 20 mM KCl, 0.2 mM EDTA, 0.5 mM DTT, 0.5 mM PMSF) followed by slowly adding 1 vol of high-salt buffer (20 mM Hepes, pH 7.6, 25% glycerol, 1.5 mM MgCl₂, 800 mL KCl, 0.2 mM EDTA, 0.5 mM DTT, 0.5 mM PMSF) at 4° C. for 30 min with agitation for nuclear protein extraction. The nuclear extracts were separated from genomic DNA and cell debris by centrifugation at 15,000 rpm/15 min at 4° C. The resulting nuclear extracts were dialyzed against 100 vol of buffer D (20 mM Hepes, pH 7.6, 20% (v/v) glycerol, 100 mM KCl, 0.2 mM EDTA, 0.5 mM DTT, 0.5 mM PMSF) for 5 h at 4° C. The dialysates were mirocentrifugated at 15,000 rpm for 10 min to remove any precipitates. The concentration of the nuclear protein was determined by Bradford assay. The resulting nuclear extracts were frozen as aliquots in liquid nitrogen and stored at −80° C.

4. In vitro Recombination Assay

The p77D3.11 plasmid used for the in vitro recombination assay was prepared in large quantity and purified using QIAGEN plasmid kits (Qiagen Inc., Chatsworth, Calif.) as described by the manufacturer. The in vitro recombination reaction was carried out in a total volume of 20 μl containing the following components: 1 μg of p77D3.11 plasmid DNA, 1-5 μg of nuclear extract protein (depending on the cell source), 0.125 mM γNTP (Promega Corp., Madison, Wis.), 20 mM Tris-HCl (pH 7.5), 3 mM MgCl₂, 1 mM spermidine (Sigma, St. Louis, Mo.), 0.5 mM dNTP (Promega Corp.), 5 mM NaCl, 10 nM Hepes, 50 mM KCl, 0.1 mM EDTA, 0.75 mM DTT and 10% glycerol. The reaction mixtures were incubated for 16 to 44 h 25° C. unless otherwise noted.

To test the effects of transcription through the S region DNA on in vitro recombination, varying amount of T7 and Sp6 RNA polymerases (Promega Corp.) were included in the reaction mixture between 0 to 60 min at 37° C. before the nuclear extracts were added.

Reactions were stopped by adding 80 μl of H₂O and extracted with phenol to remove the nuclear extract protein followed by plasmid DNA precipitation. The precipitates were washed with 70% ETOH once and the resulting precipitated DNA dissolved in 5 μl or H₂O, of which 2 μl of DNA was transformed for calculation of the white colonies vs the blue colonies. In the cases in which transformants were too many to count, DNA was diluted and the transformation was repeated. For calculation of the plasmid recovery rates, total number of transformnants were scored based on DNA dilution factors.

5. Transformation

Transformation of plasmid DNA into Escherichia coli DH10 strain (BRL) was carried out by using Gene Pulser (Bio-Rad Laboratories, Hercules, Calif.) with 0.1 c cuvettes at 1.5 kV, 200 ohm, and 25 μF. Each transformation used 2 μl of DNA and 50 μl of electro-competent cells prepared according to the BRL Cell-Porator Manual. The transformants were immediately complemented with 1 ml of SOC medium without antibiotics and incubated for 1 h at 37° C. with shaking. The transformants were placet on LB agar plates containing 100 μg/ml of ampicillin (for selection of ampicillin resistance), and 60 μl of 0.1 M IPTG (isopropyl-β-D-thiogalactoside) (Promega Corp.) and 20 μl of 50 mg/ml X-gal (5-bromo-4-chloro-3-indolyl-β-D-galactoside (Promega Corp.) for blue/white selection.

6. Polymerase Chain Reaction

Polymerase chain reaction was performed in a 50-μl volume/reaction with 50 mM KCl, 10 mM Tris-HCl (pH 8.4), 2.5 mM MgCl₂, 0.5 mM primer, and 2.5 U of Taq polymerase (Promega Corp.). For amplification of the Sμ-Sε rearranged products, the precipitated plasmid DNAs from one recombination assay reaction were first digested with Notl (20 U) for 2 h. Such treatment disrupts non rearranged but not Sμ-Sε-rearranged template plasmids and prevents their PCR amplification (see FIG. 8). PCR was carried out in 94° C. for 1 min, 60° C. for 1 min, and 72° C. for 2 min for 40 cycles with upstream primer Sμ1 (TCTAGACAAGGGGACCTGCTCATT) (SEQ ID NO:16) and downstream primer Sε4 (TTATCCCAGCAGAACTCAGTTTAAATCAC) (SEQ ID NO: 17). In order to achieve higher amplification specificity, second-round PCR was introduced to eliminate the possible nonspecific amplification form the first round PCR with primer Sμ2 (GAGCCTAGACTAACAGGCTGAACT) (SEQ ID NO: 7) and Sε3 (GCCCAGTTCAGTTAACCTCAAC) (SEQ ID NO: 18) (see FIG. 14C), although such second-round PCR is not necessary for the amplification of the recombinational products mediated by nuclear extracts.

7. Cloning of PCR Products

The pooled PCR products from a first- and second-round amplification were precipitated and directly cloned and directly cloned by TA cloning into PBK-CMV vectors (Stratagene, San Diego, Calif.) as described previously (Zhang et al., J. Immunol. 154:2237-2247 (1995)). The clones that contained inserts (white colonies) were enriched to master plates for screening of the positive clones to pSμ and pSε by in situ hybridization. The clones that hybridized to both Sμ and Sε probes were subjected to restriction endonuclease digestion and DNA sequencing analysis.

8. In situ Hybridization

The transformed white colonies were transferred to nitrocellulose membranes (Nytran, Schleichner & Schuell Inc., Keene, N.H.). The membranes were then placed onto a Whatman 3-MM paper impregnated with 10% SDS for 5 min, denatured with 0.5 N NaOH, 1.5 M NaCl for 5 min, neutralized with 1.5 NaCl, 0.5 M Tris-HCL (pH 7.4) for 5 min, and finally rinsed in 2×SSC for 5 min. The membranes were air-dried for 30 min and baked at 80° C. for 2 h followed by Southern blot hybridization (see below).

9. Southern Blot Hybridization

DNA samples of either restriction endonuclease-digested plasmid DNAs or PCR products were electrophoresed on 1% agarose gel (NuSievc and Seakem agarose 1:1 mixture, FME Biproducts, Rockland, Me.) in 0.5 c TBE buffer. DNA was then transferred to nylon membranes (Nytran, Schleicher & Schuell Inc.) in 0.4 M NaOH and blots were analyzed by hybridizing with random-labeled DNA probe. Blots were prehybridized for 2 h at 68° C. in 5×SSPE Denhardt's solution, 0.5% SDS, and 250 ng/ml of salmon sperm DNA. Hybridization was carried out overnight at 72° C. for the Sμ and Sε probes and at 68° C. for the lacZ′ gene probe. The blots were then washed for 20 min at room temperature with 2×SSC plus 0.1% SDS and then twice more at 70° C. with 0.2×SSC plus 0.1% SDS for 20 min.

10. DNA Sequencing

Nucleotide sequences of the inserts and recombined regions were determined by the standard dideoy chain termination method using a kit purchased from USB (USB, Cleveland, Ohio). Sp6 and T7 primers as well as PCR primers and synthesized oligonucleotides were used as sequencing primers as described (Li et al., Mol. Immunol. 34:201-208 (1997)).

b. Results

1. In vitro Ig Switch Region DNA Recombination Assay

A model plasmid p77D3.11 that contained a portion of human Sμ (0.91 kb) and Sε (1.25 kb) sequences with an intact lacZ gene (referred to as lacZ′) interposed between them in the vector pCR II backbone (FIG. 8) was constructed as the basis for the in vitro system of the present invention to detect Ig switch-region DNA recombination to be mediated by cell-free nuclear extracts. This construct makes it possible to monitor DNA recombination between Sμ and Sε by blue/white selection. Recombination deletes the lacZ′ gene, resulting in the formation of white colonies, whereas the non-recombined plasmid gives blue colonies as the interposed lacZ′ gene remains intact (FIG. 8). The frequency of the Sμ-Sε DNA recombination could be calculated by scoring the white vs blue colonies for different conditions from the same transformation. As the DNA fragments in the constructs were in their normal transcriptional orientation and under the control of bacteriophage T7 RNA polymerase, the construct could be used to study the effects of transcription through switch-region DNA sequences on in vitro recombination. This approach is not able to detect recombination events within the Sμ (or Sε) alone, as such recombinants would not delete the lacZ′ gene.

Restriction endonuclease analysis of the resulting white colonies using XbaI plus EcoRI can reveal recombination between various elements (e.g., Sμ-Sε, Sμ-lacZ, lacZ-Sε) as schematically diagrammed in FIG. 8. Recombination between Sμ and Sε in the model plasmid generates a single digested product when mapped with XbaI+EcoRI, whereas the non-recombined plasmids give three visible products of 0.3, 1.1, and 0.25 kb (also see FIG. 12). Recombination between Sμ and the lacZ′ gene would result in two or three digested products as indicated. Recombination between the lacZ′ gene and Sε would generate a single or two digested products depending on where the recombination site occurs. If the recombination occurs upstream of the EcoRI and/or XbaI sites of the lacZ′ gene, a single digested product would be detected while recombination occurring downstream of the EcoRI and/or XbaI sites of the lacZ′ gene would generate two digested products. Recombination between Sμ (or Sε) and the plasmid DNA sequence would result in loss of the EcoRI site in the 3′ end of Sε (or XbaI site in the 5′ end of Sμ). Digestion with EcoRI+XbaI would only linearize the circular plasmid due to the single remaining restriction site. Plasmids remaining supercoiled after digestion with EcoRI+XbaI would indicate that recombination has taken place between the vector sequences with the loss of the EcoRI and XbaI sites. Recombination between the lacZ′ gene and vector sequences (resulting in deletion of the entire Sμ or Sε fragment) would also generate a single digest product that is distinguished from that derived from Sμ-Sε recombination by Southern blot analysis with a lacZ′ gene sequence probe.

2. Ig S-S-region DNA Recombination can be Mediated by Cell-free Nuclear Extracts

To test whether Ig switch-region DNA recombination could be achieved in vitro with cell-free nuclear extracts, plasmid p77D3.11 was incubated with the nuclear extracts from human tonsillar B cells that presumably contained the putative Ig class switch recombinase because germinal center B cells in the human tonsil have been demonstrated to contain active Ig class switch recombination activity (Liu et al., Immunity 4:241-250 (1996)). The data presented in FIG. 10A show that the recombination ratio was always less than 0.1% (0.017-0.101%, n=10) in the absence of nuclear extracts. Thus, the spontaneous recombination between Sμ and Sε was very rare when the vector propagated in the host bacterial strain DH10B, a strain that has been frequently employed for assaying in vitro switch recombination (Ott et al., EMBO J. 6:577-584 (1987); Lcung and Maizels, Proc. Natl. Acad. Sci. USA 89:4154-4158 (1992); Li et al., 1997, supra). In contrast, the recombination frequency occurring in the presence of fresh tonsillar B cell nuclear extracts was 0.895% (0.594-1.40%, n=5), a rate about 20-fold higher than that in the controls. The recombination rate was further increased when CD40-stimulated B cell nuclear extracts were used, reaching an average of 3.63% (1.12-10.56%, n=18). The recombination effect was abolished by heating the nuclear extracts at 80° C. for 30 min (FIG. 10A), suggesting that the protein components in the nuclear extracts were required for the recombination activity. Addition of EDTA (10 mM) to the recombination reaction almost completely blocked the activity, indicating that Mg²⁺ was required for recombination (FIG. 10A). ATP was necessary for the assay as its depletion resulted in the recombination rates close to the background (0.25%, n=7) (FIG. 10A). Kinetic determination of the switch recombination activity shows that the activity was dose-dependent at nuclear protein concentrations from 1 to 5 μg/reaction (FIG. 11A). The reactions were incubation temperature-dependent, with optimized temperature at 25° C. (FIG. 11B), and incubation time-dependent, with best results achieved between 16 and 44 hours (FIG. 11C). Overall, these results indicate that recombination between Sμ and Sε could be achieved by nuclear extracts from B cells in vitro.

The recombination activity from other types of primary cells and cell lines-was also investigated. Nuclear extracts from primary human tonsil T cells showed lower (0.52%) but consistent recombination activity under conditions comparable to B cells (e.g., the protein concentration from T cells is the same as that from B cells (FIG. 9A)). Such lower recombination activity also can be detected, although varied, in all the cell lines tested so far, including lymphoid cells (2C4/F3, JY, Ramos, A11, AF-10, Jurkat, I-29) and non-lymphoid cells (293, CHO, HeLa) (FIG. 9B). The recombination frequencies generated from these various cell lines, however, cannot be directly compared to each other or to primary B and T cells. The nuclease activity in these cell lines was much higher than in primary B cells and greatly different from each other since the same concentration of the nuclear protein from cell lines and from primary B cells (5 μg/reaction) almost completely degraded the input plasmids (data not shown). Overall, the results suggested that the recombination activity was not restricted but significantly higher in primary B cell stimulated with CD40.

Results from mapping of the recombinant colonies are summarized in FIG. 8. Of a total 307 white colonies obtained from 12 independent experiments, 269 colonies (87.6%) gave a single band with XbaI plus EcoRI digestion, indicating that the recombination in these colonies occurred between Sμ and Sε regions, as occurs with CSR. FIG. 12 shows one mapping experiment in which 29 randomly picked recombined clones were analyzed by XbaI+EcoRI digestion. In this experiment, all the clones gave a single digested band (FIG. 12A) and all but one hybridized to either Sε (FIG. 12B) or Sμ probes (FIG. 12C) but not the probe containing lacZ′ gene sequences (FIG. 12D). Those clones had undergone the Sμ-Sε recombination in this in vitro assay.

3. IgS-S DNA Recombination Catalyzed by Nuclear Extracts Occurs Prior to Transformation

To definitively show that the S-S recombination is mediated by the nuclear extracts in vitro rather than during or following bacterial transformation, PCR analysis was performed on the Sμ-Sε recombination products following nuclear extract reaction but prior to bacterial transformation. The precipitated plasmid DNA resulting from incubation with or without nuclear extracts was used as DNA templates for PCR amplification. To optimize the opportunity to amplify Sμ-Sε-recombined products, the template DNA was digested with the restriction endonuclease NotI prior to PCR amplification as the majority of the input plasmids were not rearranged. Such treatment disrupts non-rearranged but not Sμ-Sε-rearranged template plasmids (FIG. 7). As the amplification products appeared as a smear rather than a set clear band when resolved on agarose gels (data not shown), the PCR products were directly cloned by TA cloning methods from the whole PCR mixture (Zhang et al., 1994, supra). The resulting clones were screened by differential in situ hybridization with Sμ and Sε probes (FIG. 13). Seventeen out of 357 clones screened hybridized to both Sμ and Sε probes (FIG. 8), showing that they represented Sμ-Sε-rearranged products that were further confirmed by sequence analysis (FIG. 14B). By using the amplification strategy for detection of the looped-out circular DNA products and the DNA inversion events in the constructs, the product representing deleted circular DNA and DNA inversion were also identified (Jack et al., Proc. Natl. Acad. Sci. USA 85:1581-1585 (1988); Laffan and Luzzatto, J. Clin. Invest. 90:2299-2307 (1992)) through cloning and sequencing analysis (data not shown). These results demonstrate that recombination of human Sμ and Sε DNA is catalyzed by the cell-free nuclear extracts from human B cells when added to the in vitro assay.

4. Ig S-S Recombination Mediated by Nuclear Extracts in vitro is Non-homologous Recombination

One characteristic feature of Ig class switch recombination is that deletional recombination between two involved S regions is region specific but site non-specific; i.e. it falls in the category of “illegitimate” recombination (Dunnick et al., Nucleic Acids Res. 21:365-372 (1993)). To show that the recombination sites employed following nuclear extract-driven recombination demonstrate this feature, we sequenced the recombination junctures of recombined clones. Thirty-two randomly selected colonies that hybridized to both Sμ and Sε probes from the in vitro recombination assay and 17 colonies derived from direct PCR amplification were sequenced. The breakpoints were defined by alignment of the clone sequences with the Sμ and Sε sequences in plasmid p77D3.11. The sequences around the recombination junctions from 10 clones derived from recombination assay and 10 clones from PCR amplification are shown in FIGS. 13A and 13B, respectively. The homology between Sμ and Sε in these sequence identified clones from both recombination assay and PCR assay varies from a homology of 15 (clone SR.23-10) nucleotides to 0 (e.g., clones R5.22-10, R5.29-22, SR6.16-13). Clones that contained unmatched sequences in the breakpoint to both Sμ and Sε are also presented (clones R6.4-39, SR9.24-20, and SR28-27). Thus, although clones derived from the in vitro recombination assay often share a short stretch of homology for Sμ and Sε in the junctional sites as seen in in vivo CSR, homology between Sμ and Sε was not required for switch-region DNA recombination in our system. The frequently observed short homologous stretches between Sμ and Sε at the recombination junctions likely reflect the internal features of the Sμ and Sε constructs used to make p77D3.11, with its tandemly repetitive sequences GGGCT, GGGGT, and GAGCT densely distributed throughout the Sμ and Sε segments.

5. Ig S-S DNA Recombination in vitro Preferentially Targets Tandemly Repetitive Sequences

The sequences recombination breakpoints assigned to Sμ and Sε from both the recombination assay and the PCR assay are summarized in FIG. 14C. Strikingly, all 41 recombination breakpoints are located in the tandemly repeated regions in Sμ and Sε None of them occurred in the lacZ′ gene or 5′ to Sμ (FIG. 14C). Although the recombination breakpoints in the PCR-generated clones may represent selection biased by the PCR assay itself and/or cloning procedures employed, the targeting of breakpoints to the tandem repetitive Sμ and Sε regions in the recombination assay-generated clones demonstrates that the recombination activity detected in the in vitro recombination assay preferentially targets the tandemly repetitive S region sequences. Otherwise some of the recombination sites would be expected to scatter in the lacZ′ gene and/or regions 5′ to Sμ.

To further examine the sequence requirements for recombination activity in the present in vitro assay, the entire Sε sequences in p77D3.11 were replaced with an 0.9-kb fragment derived from a sequence immediately 5′ to Sε, a sequence that does not contain typical G-rich, tandemly repetitive sequences. The modified construct, p77D3.11a, when compared with p77D3.11, generated a >10-fold lower recombination frequency (0.20% with p77D3.11a vs. 3.63% with p77D3.11 in average). The recombination rate with p77D3.11 a is barely above the highest background level (0.1%). These results further support the observation that recombination activity in this system preferentially targets G-rich, tandemly repetitive S sequences as recombination substrates.

6. Transcription Through the S Region is Not Required for Ig S DNA Recombination in the in vitro Recombination System

It has been suggested that transcription through the targeted S region is a prerequisite for in vivo switch recombination. All the results shown were obtained under the conditions without transcription activity through the S region (i.e., without addition of bacterial T7 RNA polymerase) in the recombination reaction. Thus, the in vitro recombination system does not require transcription through the S region fir Sμ-Sε recombination. While p77D3.11 contained in the Iμ promoter and μ enhancer sequences, (Eμ fragment) that initiated Iμ transcription in vivo (35, 36), this was not required for Sμ-Sε recombination in the system as the deletion of the Eμ fragment from p77D3.11 did not significantly alter the recombination frequency nor the nature of the recombination events observed (data not shown). The effects of transcription through S regions on recombination in the present in vitro recombination system have been tested with bacterial T7 or Sp6 RNA polymerases and the putative relationship between transcription activity and switch recombination frequencies have not been firmly established in this system (data not shown).

c. Discussion

The Sμ-Sε DNA recombination obtained in the in vitro assay of the present example possessed several key features of Ig S-S recombination in vivo. First, the sequences around the recombination breakpoints revealed that the Sμ-Sε recombination was non-homologous even though tandemly repetitive sequences are densely distributed in both Sμ and Sε fragments in the recombination substrate. Second, there was restricted distribution of the recombination breakpoints to G-rich S region sequences with the requirement for switch rather than nonswitch-region G-rich DNA, indicating that this system has preferential targeting of G-rich switch-region DNA sequences. Third, enhancement of S-S recombination by the nuclear extracts from CD40-driven B lymphocytes agrees with the requirement of the CD40 stimulation on B cells for Ig class switching. Taken together, these results strongly suggest that the recombination activity assayed in the in vitro recombination system reflects the normal processes involved in the Ig CSR in vivo. The biochemical nature (makeup) of the Ig switch recombinase activity, which is believed to take part in the process of switching to all Igs including IgE is largely unknown. The present in vitro recombination system that can specifically detect and quantify Ig CSR will provide a tool for characterizing the nature of the Ig switch recombination process, determining the components involved, and isolating the putative switch recombinase and/or the components participating in this process.

There are notable differences between the in vitro S region DNA recombination and the in vivo Ig CSR. Switch recombination in vivo requires orchestration of at least two independent intracellular processes, e.g., Ig germline transcription that is though to render the S region DNA accessible for the targeting of the putative switch recombinase and deletional switch region DNA recombination. The in vitro S DNA recombination system employs a small model construct and nuclear extracts in the absence of the gemline transcripts or the need for transcriptional activity through S region DNA. It was not surprising that transcription through the S region would not be required, since the plasmid S region DNA is likely to be already in an accessible configuration for switch recombinase activity in such artificial constructs.

As G-rich switch-region DNA undergoes rearrangement in bacteria, the possibility of Sμ-Sε recombination during the bacterial transformation process and the fidelity of the in vitro recombination mediated by nuclear extracts required special scrutiny. Plasmid 77D3.11, which contains several inserted DNA fragments including Sμ and Sε, is stably propagated in the bacteria competent cells with a recombination background below 0.1% (FIG. 8). This property was required to ensure that the measured recombination rates were not caused by bacterial recombination during the transformation process. To conclusively demonstrate that the cell-free nuclear extracts were driving the recombination process in vitro, recombination products were amplified and cloned through a PCR-based strategy, without having been transformed into bacteria. The products so analyzed showed that S region recombination events resulted from the actual in vitro recombination system.

The nature of the S region DNA dependency and non-homologous recombination in the in vitro recombination system distinguishes it from previously described nuclear extracts-driven homologous and illegitimate recombination systems (Tsukamo et al., Nature 388:900-903 (1997); Lopez et al., Nucleic Acids Res. 15:6813-6826 (1987); Pfeiffer and Vielmetter, Nucleic Acids Res. 16:907-924 (1988); Lopez et al., Nucleic Acids Res. 20:501-506 (1992); Thacker et al., Nucleic Acids Res. 20:6183-6199 (1992)). The preferential targeting of G-rich, tandemly repetitive sequences in Sμ region by the nuclear extract recombination activity in the assay resembles primary switch recombination events as defined by the analysis of switch circular DNA that preferentially targets the S region sequences (Dunnick et al., 1993, supra; Zhang et al., 1994, supra; Rabbitts et al., 1983, supra; Matsuoka et al., Cell 62:135-144 (1990); Von Schwedler ct al., Nature 345:452-455 (1990)). This is significantly different from the recombination sites defined from Ig class switched B cell lines (Dunnick et al., 1993, supra; Matsuoka et al., 1990, supra) and some of the switch recombination assays reported (Leung and Maizels, 1992, supra; Leung and Maizels, Mol. Cell. Biol. 4450-1458)1994); Li et al., 1997, supra; Daniels and Lieber, Nucleic Acids Res. 23:5006-5011 (1995)), in which the switch recombination breakpoints were frequently located in non-G-rich, tandemly repetitive sequences outside of the S region.

Characterization of the types of recombined products from the present recombination substrate further defined the events observed as representative of the outcomes occurring in vivo. Definition of both excised circular DNA and DNA inversion products (data not shown) in this system provides strong evidence that the S-S DNA recombination is mediated through the predicted processes (Jack et al., 1988, supra; Laffan and Luzzaato, 1992, supra). These findings also demonstrate that the looping-out, cleavage, and religation processes occurring in the in vitro recombination are tightly coupled processes that are likely being mediated by the switch recombinase complexes rather than occurring by random cleavage and ligation reaction mediated by nonspecific endonuclease and DNA ligase that might exist in the nuclear extracts. The appearance of nucleotides unmatched to the template switch region DNA (Sμ an Sε) at the recombination breakpoints in some clones (clones R6.4-39, SR9.24-20, and SR9.28-27) is a phenomenon that is frequently observed at switch recombination breakpoints generated in vivo. This suggests that the proposed in vivo “error-prone” mechanism also operates appropriately in the present in vitro recombination assay (Dunnick and Hertz, 1993, supra).

The recombination activity detected in the present assay from primary B cells as well as non-B cells indicates that, although it is not specifically restricted in B cells, the recombination activity is higher in primary B cells; more significantly, it is up-regulated by CD40 stimulation. The non-restricted expression of the recombination activity suggests that the actual Ig CSR in vivo may be controlled at a transcriptional level other than in the S DNA recombination level, as the “accessibility” model suggested (Esser and Radbruch, Annu. Rev. Immunol. 8:717-735 (1990); Coffinan et al., Adv. Immunol. 54:229-270 (1993); Stavnezer, J., Adv. Immunol. 61:79-90 (1996); Dunnick and Hertz, 1993, supra; Harrima et al., Annu. Rev. Immunol. 11:361-384 (1993); Stavnezer-Nordgren and Sirlin, EMBO J. 5:95-102 (1986); Snapper et al., Immunity 6:217-223 (1997)), since germline transcription correlated with the specificity of a given isotype switch. Alternatively, switch-region DNA-specific recombination machinery (presumably the switch recombinase complex) may share components with the recombinase activity that participates in non-switch-region DNA cells; for example Ku protein and DNA-dependent protein kinase have been shown to be involved in VDJ recombination and CSR (Rolink et al., Immunity 5:319-330 (1996); Zelazowski et al., J. Immunol. 159:2559-2562 (1997); Manis et al., J. Exp. Med. 187:2081-2088 (1998); Casellas et al., EMBO J. 17:24-4-2411 (1998)). Under these circumstances, the recombinase activities are expected to be non-B cell restricted in the present in vitro recombination assay.

Overall, the results presented here demonstrate that Ig switch-region DNA recombination can be accomplished in vitro using a model switch vector and cell-free nuclear extracts. Characterization of the recombination products demonstrated that the recombination process had the characteristics of Ig isotype switching, as it was (i) switch region sequence specific, (ii) non-homologous recombination, and (iii) enhanced y CD40 stimulation. Transcription through the S region DNA was not required for recombination in this system. This in vitro system for Ig switch-region DNA recombination using cell-free nuclear extracts will permit further dissection of the events involved in IgE class switch recombination.

What is claimed is:

1. An isolated nucleic acid molecule comprising
(a) a first class switch region (S₁) nucleotide sequence of an upstream immunoglobulin locus under transcriptional control of a first promoter;
(b) a second class switch region (S₂) nucleotide sequence of an immunoglobulin locus downstream of said upstream Ig locus under transcriptional control of a second promoter, wherein said S₂ sequence serves as a region-specific substrate for class switch recombination (CSR);
(c) a reporter gene nucleotide sequence encoding a reporter molecule, interposed between said S₁ and S₂ sequences in reverse transcriptional orientation, and
(d) a promoter, downstream of said nucleotide sequence encoding said reporter molecule, allowing the expression of said reporter molecule only following CSR between said S₁ and S₂ sequences.

2. The nucleic acid molecule of claim 1 wherein said S₁ is an Sμ sequence and said S₂ is an Sγ2 sequence.

3. The nucleic acid molecule of claim 1 wherein said S₁ is an Sμ sequence and said S₂ is an Sε sequence.

4. The nucleic acid molecule of claim 2 wherein said S₁ and S₂ sequences are G-rich switch region DNA sequences.

5. The nucleic acid molecule of claim 3 wherein said S₁ and S₂ sequences are G-rich switch region DNA sequences.

6. The nucleic acid molecule of claim 1 wherein said nucleic acid in part (c) and said promoter in part (d) are under control of an internal ribosome entry site (IRES).

7. The nucleic acid molecule of claim 1 wherein said nucleic acid in part (c) encodes a Green Fluorescent Protein (GFP) molecule.

8. The nucleic acid molecule of claim 1 wherein said nucleic acid in part (c) encodes a reporter molecule selected from the group consisting of β-galactosidase, luciferase, and secreted alkaline phosphatase (SEAP).

9. The nucleic acid molecule of claim 1 wherein said first and second promoters are non-inducible constitutive promoters.

10. The nucleic acid molecule of claim 9 wherein said first promoter is a CMV promoter.

11. The nucleic acid molecule of claim 9 wherein said second promoter is an SV promoter.

12. An isolated nucleic acid molecule comprising
(a) a human Sμ nucleotide sequence under control of a CMV promoter;
(b) a human Sγ₂ nucleotide sequence under control of an SV promoter;
(c) an RSV LTR enhancer/promoter and GFP gene under control of an internal ribosome entry site (IRES), interposed between said Sμ and Sγ₂ sequences, in reverse transcriptional orientation,
(d) a 5′ splicing donor site from human β-globulin gene, 3′ of said Sμ sequence; and
(e) a 3′ splicing acceptor site and Cε1 exon, 3′ of said Sγ₂ sequence.

13. The nucleic acid molecule of claim 12 further comprising a nucleic acid fragment of a cytokine-inducible promoter for Ig germline transcription, 5′ of said CMV promoter.

14. The nucleic acid molecule of claim 13 wherein said cytokine-inducible promoter is an IL-4 inducible Iε promoter.

15. The nucleic acid molecule of claim 12 selected from the group consisting of XF-1, XF-5a, XF-8, XF-2a, XF-2b, XF-6a and XF-6b.

16. A switch vector comprising a nucleic acid molecule of claim 1.

17. A switch vector comprising a nucleic acid molecule of 12.

18. A recombinant host cell stably transfected with the switch vector of claim 16.

19. A recombinant host cell stably transfected with the switch vector of claim 17.

20. The host cell of claim 18 which is a mammalian cell.

21. The host cell of claim 20, which is a Chinese Hamster Ovary (CHO) cell.

22. The host cell of claim 20 which is a primary human B cell.

23. A method of monitoring immunoglobulin (Ig) class switch recombination (CSR), comprising
(a) providing a switch vector comprising
(i) a first class switch region (S₁) nucleotide sequence of an upstream Ig locus under transcriptional control of a first promoter;
(ii) a second class switch region (S₂) nucleotide sequence of an Ig locus downstream of said upstream Ig locus under transcriptional control of a second promoter, wherein said S₂ sequence serves as a region-specific substrate for CSR;
(iii) a reporter gene nucleotide sequence encoding a reporter molecule interposed between said S₁ and S₂ sequences in reverse transcriptional orientation, and
(iv) a promoter, downstream of said nucleotide sequence encoding said reporter molecule, allowing the expression of said reporter molecule only following switch recombination between said S₁ and S₂ sequences;
(b) stably transfecting a mammalian cell with said switch vector; and
(c) monitoring the expression of said reporter molecule in said mammalian cell, wherein such expression indicates CSR.

24. The method of claim 23 wherein said mammalian cell is a primary B cell or a B cell line.

25. The method of claim 24 wherein said B cell line is a human B lymphoma cell line.

26. The method of claim 25 wherein said cell line contains a single copy of said switch vector.

27. The method of claim 23 wherein said reporter molecule is Green Fluorescent Protein (GFP).

28. The method of claim 27 wherein CSR is monitored by fluorescence microscopy.

29. The method of claim 28 further comprising the step of quantifying CSR.

30. The method of claim 29 wherein said CSR is quantified by flow cytometry.

31. The method of claim 29 wherein said first promoter is a CMV promoter.

32. The method of claim 29 wherein said second promoter is an SV promoter.

33. The method of claim 31 wherein said switch vector further comprises a cytokine-inducible promoter for Ig germline transcription 5′ of said CMV promoter.

34. The method of claim 33 wherein said cytokine-inducible promoter is an IL-4 inducible Iε promoter.

35. The method of claim 34 further comprising the step of culturing said cells in the presence of IL-4 and/or a stimulator of CD40 activity prior to monitoring CSR.

36. The method of claim 35 wherein said stimulator of CD40 activity is an anti-CD40 monoclonal antibody (mAb) or a CD40 ligand.

37. The method of claim 35 further comprising the step of exposing said cells to a candidate molecule, and determining the effect of said candidate molecule on GFP expression.

38. A method of monitoring immunoglobulin (Ig) class switch recombination (CSR) comprising
(a) providing a switch vector comprising, under transcriptional control of a promoter and in natural transcriptional orientation,
(i) a first class switch region (S₁) nucleotide sequence of an upstream Ig locus;
(iii) a second class switch region (S₂) nucleotide sequence of an Ig locus downstream of said upstream Ig locus; and
(iv) a reporter gene nucleotide sequence encoding a reporter molecule, interposed between said S₁ and S₂ sequences;
(b) incubating said switch vector with a cell-free nuclear extract from Ig-producing cells or cells with Ig-producing potential; and
(c) detecting deletion of said reporter gene.

39. The method of claim 38 wherein said first class switch region sequence (S₁) is an Sμ sequence and said second class switch region sequence (S₂) is an Sε sequence.

40. The method of claim 38 wherein deletion of said reporter gene is detected following transformation of said switch vector into a recombinant host cell.

41. The method of claim 40 wherein said recombinant host cell is a prokaryotic cell.

42. The method of claim 41 wherein said prokaryotic cell is an E. coli cell.

43. The method of claim 40 wherein said reporter gene is a lacZ gene.

44. The method of claim 43 wherein deletion of said reporter gene is detected by counting the white colonies obtained after transformation, in the presence of isopropyl-β-D-thiogalactoside (IPTG) and 5-bromo-4-chloro-3-indolyl-β-D-galactoside (X-gal).

45. The method of claim 38 wherein said Ig-producing cells are B lymphocytes.

46. The method of claim 45 wherein said B lymphocytes are of human origin.

47. The method of claim 38 wherein said Ig-producing cells are primary B cells stimulated with CD40.

48. The method of claim 38 wherein said S₁ and S₂ comprise G-rich, tandemly repetitive sequences.
